(19) United States
(12) Patent Application Publication
(10) Pub. No.: US 2016/0161499 A1
(43) Pub. Date: Jun. 9, 2016

(54) SENSITIVE DIAGNOSTIC ASSAY FOR INCLUSION BODY MYSITIS

(71) Applicants: THE BRIGHAM AND WOMEN'S HOSPITAL, INC., Boston, MA (US); CHILDREN'S MEDICAL CENTER CORPORATION, Boston, MA (US)

(72) Inventor: Steven GREENBERG, Newton, MA (US)

(73) Assignees: THE BRIGHAM AND WOMEN'S HOSPITAL, INC., Boston, MA (US); CHILDREN'S MEDICAL CENTER CORPORATION, Boston, MA (US)

(21) Appl. No.: 14/907,625

(22) PCT Filed: Aug. 12, 2014
(86) PCT No.: PCT/US14/50626

§ 371 Date: Jan. 26, 2016

Related U.S. Application Data
(60) Provisional application No. 61/865,219, filed on Aug. 13, 2013.

(51) Int. Cl. G01N 33/68

(52) U.S. Cl. CPC G01N 33/6854; G01N 2800/10; G01N 2800/50

(57) ABSTRACT

Assay methods, kits and systems for an improved sensitive diagnosis of inclusion body myositis (IBM) are provided. Assays, kits and systems involve detecting the presence and/or level of a combination of more than one autoantibody isotypes that are reactive against a cytosolic 5′-nucleotidase 1A protein (CN1A), or a cytosolic 5′-nucleotidase 1B protein (CN1B), or a CN1B isoform thereof, or a peptide fragment thereof, an isolated peptide thereof or a fusion protein comprising an isolated peptide of CN1A or CN1B. IBM patients have differing patterns of anti-cN1A/cN1B autoantibody isotypes. Therefore, testing a combination of such isotypes provide an improved and sensitive diagnostic assay.

### CROSS REFERENCE TO RELATED APPLICATIONS

This application claims benefit under 35 U.S.C. §119(e) of U.S. Provisional No. 61/865,219, filed Aug. 13, 2013, the contents of which are incorporated herein by reference in their entirety.

### TECHNOLOGICAL FIELD

Embodiments of the technology described herein relate to the development of highly sensitive and non-invasive diagnostics for the muscle disorder inclusion body myositis (IBM).

### BACKGROUND

Myositis is one form of inflammatory myopathy involving chronic inflammation of the skeletal muscles. It is a rare disease in which the immune system chronically inflames the body's muscle tissue. The etiology of the immune system's attack is currently unknown. Persistent inflammation progressively weakens the muscles, causing muscle soreness, joint pain and fatigue.

Myositis can take several forms and usually develops slowly over time and can range in severity from mild to debilitating or worse. The three major types of myositis are dermatomyositis (DM), polymyositis (PM), and inclusion body myositis (IBM). PM affects skeletal muscles on both sides of the body and mostly affects people between the ages of 31 and 60. Progressive muscle weakness often leads to difficulty swallowing (dysphagia), rising from a sitting position, climbing stairs, lifting objects, or reaching overhead. People with PM may also experience arthritis, shortness of breath, and heart arrhythmias.

In addition to progressive muscle weakness, patients with DM can have a characteristic skin rash that precedes or accompanies progressive muscle weakness. The rash looks patchy, with purple or red discolorations, and is characteristically found on the eyelids and on skin overlying joints of knuckles, elbows, knees, and toes. Red rashes are also found on other locations such as the face, neck, shoulders, upper chest, and back. The rash sometimes occurs without obvious muscle involvement.

IBM is characterized by gradual (over months or years) progressive muscle weakness and wasting that affect both proximal and distal muscles. IBM affects individuals in a variety of different ways from the age of onset. Symptoms of the disease usually begin after the age of 50, although the disease can occur earlier. Muscle weakness may affect only one side of the body. Falling and tripping are usually the first noticeable symptoms of IBM. For some individuals the disorder begins with weakness in the wrists and fingers that causes difficulty with pinching, buttoning, and gripping objects. There may be weakness of the wrist and finger muscles and atrophy (thinning or loss of muscle bulk) of the forearm muscles and quadriceps muscles in the legs. Difficulty swallowing occurs in approximately half of IBM cases. Unlike PM and DM, IBM occurs more frequently in men than in women. In addition to physical symptomatic changes, the muscles of IBM patients have small degenerative structures that with special staining appear as holes called vacuoles in the affected muscle fibers.

Diagnosing IBM can be challenging. Circulating autoantibodies, such as anti-Jo and anti-SRP, have been identified in PM and DM, and have been used to aid both in diagnosis and the treatment of these two conditions. Recently, IgG autoantibodies against cytoplasmic 5′ nucleotidase (cN1A) have been reported to be associated with IBM, hence a simple non-invasive blood test for the presence of anti-cN1A IgG autoantibodies can help identify and diagnose IBM patients. However, not all IBM individuals tested positive for the presence of anti-cN1A IgG autoantibodies. The reported sensitivity of such a test based on detecting the anti-cN1A IgG autoantibody for identifying and diagnosing IBM patients is approximately ˜50%. Therefore, there is at least a 50% chance that an individual having IBM may not be identified using the test that detects for IgG autoantibody against cN1A. Consequently, this low sensitivity can lead to initial misdiagnosis and a definitive diagnosis is delayed.

IBM is often misdiagnosed as some other inflammatory myopathy, usually PM. For example, a course of prednisone is typically completed with no improvement and eventually IBM is suspected. IBM weakness comes on over months or years and progresses steadily, whereas PM has an onset of weeks or months. Nowadays, differential diagnosis of IBM involves systematically ruling out other possible causes such PM, DM, chronic inflammatory demyelinating polyradiculoneuropathy, Duchenne muscular dystrophy, and myasthenia gravis etc. Currently the only definitive test for IBM is a muscle biopsy to confirm the physical changes in the affected muscles. More sensitive, non-invasive diagnostic tests are needed.

### SUMMARY

Embodiments of the technology described herein are based on the discovery of a variety of circulating autoantibody isotypes that are specific to inclusion body myositis (IBM) and not dermatomyositis (DM) and polymyositis (PM). Healthy patients free of any inflammation conditions and patients with other myositis, e.g., PM and DM, do not have any such autoantibody isotype.

IBM is a poorly understood autoimmune and degenerative muscle disease. Recent studies have identified circulating IgG, IgA and IgM autoantibodies against cytoplasmic 5′ nucleotidase (cN1A) in IBM patients. The use of a combination of more than one anti-cN1A autoantibody isotypes provides a non-invasive diagnostic test for IBM with greater sensitivity compared to a similar blood diagnostic test that just focuses only on anti-cN1A IgG autoantibody isotype.

The objective here is to provide improved non-invasive diagnostic methods, assays, kits and systems for diagnosing the likelihood of IBM with greater sensitivity and with no loss of specificity. The non-invasive diagnostic methods, assays, devices, kits and systems are also useful for evaluating treatment or therapeutic drug effectiveness and also IBM prognosis and progression.

Accordingly, in one embodiment, provided herein is an assay for diagnosing the likelihood of IBM, the assay comprises (a) providing a biological sample from a subject in need of diagnosing, and (b) measuring for a detectable presence or an absence or a level of at least two antibody isotypes that are reactive against a cN1A or a protein fragment thereof, or a cytosolic 5′-nucleotidase 1B (cN1B) protein or isoforms thereof or a protein fragment thereof for diagnosing or detecting IBM in a subject wherein the antibody isotypes are selected from the group selected from IgG, IgM, IgA, IgD, and IgE.

In another embodiment, provided herein is an assay for evaluating the efficacy of a treatment for a patient having IBM, the assay comprising (a) measuring for an absence or a presence or a level of at least two antibody isotypes that are reactive against a cN1A, or a c1B protein or isoforms thereof in at least two biological samples that are chronologically separated apart, e.g., at a first time point and a second time point; and (b) comparing the absence/presence or levels of the autoantibody isotypes in the at least two biological samples, wherein the antibodies isotypes are selected from the group selected from IgG, IgM, IgA, IgD, and IgE.

In one embodiment, provided herein is an assay comprising (a) measuring for an absence or a presence or a level of at least two antibody isotypes that are reactive against a cN1A, or a c1B protein or isoforms thereof in at least two biological samples obtained from a subject at different times for a prognosis evaluation of the subject with IBM or for evaluating the efficacy of a treatment in the subject with IBM, wherein the different times are a first time point and at least a second time point, wherein the second time point is after the first time point, and wherein the antibodies isotypes are selected from the group selected from IgG, IgM, IgA, IgD, and IgE; and (b) evaluating the prognosis of the subject or the treatment efficacy based on the detectable presence or the level of the at least two antibody isotypes selected in above measuring step (a).

In one embodiment of the assay described, the patient has IBM and is being treated for IBM.

In one embodiment of the assay described, the at least two biological samples that are chronologically separated apart comprises a first biological sample and a second biological sample from the patient wherein the first sample is taken at a first time point and the second sample is taken at a second time point, and the second time point is after the first time point.

In one embodiment of the assay described, when there is no detectable presence (ie., below a detection limit of the measuring method) of the tested autoantibody isotypes taken at the second time point compared to the first time point where there was a detectable presence of the autoantibody isotypes indicates that the treatment is effective and that the patient is in remission.

In alternate embodiment of the assay described, when there is continued detectable presence of the tested autoantibody isotypes taken at the second time point compared to the first time point where there was a detectable presence of the autoantibody isotypes tested indicates that the IBM in the patient is approximately the same, and indicates that the treatment is not effective.

In one embodiment of the assay described, when there is a decrease in the level of the autoantibody isotypes tested taken at the second time point compared to the first time point indicates that the treatment is effective, wherein an increase in the level of the autoantibody isotypes tested taken at the second time point compared to the first time point or the levels are approximately the same indicates that the treatment is not effective, and wherein when the level of autoantibodies in the second time point decreases to below a detection limit indicates that the treatment is effective and the patient is in remission.

In one embodiment of any one of the assays described, the assay further comprising diagnosing the likelihood of IBM based on a detectable presence or the level of the at least two antibody isotypes selected in measuring step.

In one embodiment of any one of the assays described, a detectable presence of at least two autoantibody isotypes reactive against cN1A or protein fragment thereof, or cNIB, isoforms or protein fragment thereof indicates the likelihood of IBM in the subject.

In one embodiment of any one of the assays described, the detectable presence of the at least two autoantibody isotypes is based on the detection limit of the detection method utilized. For example, the anti-cN1A or cN1B autoantibody isotypes can be analyzed in a 1D SDS PAGE gel followed by a Western blot analysis. The detectable presence of the autoantibody isotypes would be based on the detection limit of the Western blot on a 1D SDS PAGE gel.

In one embodiment of any one of the assays described, the assay has a sensitivity of at least 55%.

In one embodiment of any one of the assays described, the assay has a specificity of no less than 90%.

In one embodiment of any one of the assays described, the assay has a sensitivity of at least 55% and a specificity of no less than 90%.

In one embodiment of any one of the assays described, the subject presents at least one symptom of a muscle disease. For example, muscular weakness, rigidity, loss of muscular control, myoclonus (twitching, spasming), and myalgia (muscle pain).

In one embodiment of any one of the assays described, the muscle disease is an inflammatory myopathy. For example, polymyositis (PM), dermatomyositis (DM), and inclusion body myositis (IBM).

In one embodiment of any one of the assays described, the at least one symptom is selected from the group consisting of progressive weakness in the muscles of the wrists and fingers, progressive weakness in the muscles that lift the front of the foot, progressive weakness in the muscles at the front of the thigh (quadriceps), and weakness of the swallowing muscles.

In one embodiment of any one of the assays described herein, the subject presents symptoms of IBM, such as progressive muscle weakness, muscle pain and/or muscle fatigue

In one embodiment of any one of the assays described herein, the PM, DM, other known autoimmune disorders has been rule out in the subject, i.e., confirming that the patient is not suffering from PM, DM, or Myasthenia gravis etc.

In one embodiment of any one of the assays described, the subject in need of diagnosing has trouble with gripping, or has frequent stumbles, or has trouble swallowing due to weakness of the swallowing muscles.

In one embodiment of any one of the assays described, the subject has not had to an electromyogram and/or a muscle biopsy.

In one embodiment of any one of the assays described herein, the subject is suspected of having IBM.

In one embodiment of any one of the assays described herein, the IBM is idiopathic.

In one embodiment of any one of the assays described, the subject is being treated for IBM. In one embodiment of any one of the assay described, the treatment is experimental.

In one embodiment of any one of the assays described, the subject had previously been successfully treated for IBM and the IBM is in remission.

In one embodiment of any one of the assays described, the subject is a human.

In one embodiment of any one of the assays described, the autoantibodies are from a blood sample of the patient. Accordingly, in one embodiment of any one of the assay described, the biological sample is a blood sample. In another embodiment of any one of the assay described, the blood sample is plasma or serum.

In one embodiment of any one of the assays described, the at least two isotypes are the combinations as follows: IgG and IgM; IgG and IgA; IgG, IgA and IgM; IgA and IgM; IgG and IgD; IgG and IgE; IgD and IgM; IgE and IgM; IgE and IgA; IgD and IgA; IgE, IgM and IgA; IgD, IgM and IgA; IgD, IgE and IgM; IgD, IgE and IgA; and IgD, IgE, IgA and IgM.

In another embodiment of any one of the assays described, the at least two isotypes are the combinations as follows: IgE and IgD; IgG, IgA, and IgD; IgG, IgA, and IgE; IgG, IgM, and IgD; IgG, IgM, and IgE; IgG, IgD, and IgE; IgG, IgA, IgM, and IgD; IgG, IgA, IgM, and IgE; IgG, IgA, IgD, and IgE; and IgG, IgM, IgD, and IgE.

In one embodiment of any one of the assays described, the at least two isotypes selected in the assay are IgA and IgM.

In one embodiment of any one of the assays described, the at least two isotypes selected in the assay are IgG and IgM.

In one embodiment of any one of the assays described, the at least two isotypes selected in the assay are IgG, IgA and IgM.

In one embodiment of any one of the assays described, the at least two isotypes selected in the assay does not include IgG. In particular, the assay does not include IgG1 and/or IgG2a.

In one embodiment of any one of the assays described, IgM is one of the at least two isotypes selected.

In one embodiment of any one of the assays described, IgM is one of the at least two isotypes selected and IgG is not selected. In particular, IgG1 and/or IgG2a are not selected.

In one embodiment of any one of the assays described, the detectable presences of the at least two antibody isotypes selected indicate the likelihood of IBM in the subject.

In one embodiment of any one of the assays described, the assay further comprises comparing the level of the at least two antibody isotypes selected to respective antibody isotypes reference levels to determine the likelihood of IBM in the subject.

In one embodiment of any one of the assays described, the level of the at least two isotypes that are at least 5% over that of respective antibody isotype reference levels indicate the likelihood of IBM.

In one embodiment of any one of the assays described, the respective antibody isotype reference levels are the levels of the selected antibody isotypes that are reactive against the same cN1A, cN1B or protein fragment thereof in a biological sample of a healthy subject not having IBM, wherein the biological samples are the same for both the healthy subject and subject in need of diagnosis.

In one embodiment of any one of the assays described, the respective antibody isotype reference level are average levels of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis.

In one embodiment of any one of the assays described, the respective antibody isotype reference level are average levels plus one or two or three standard deviations of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis.

In one embodiment of any one of the assays described, the respective antibody isotype reference level are normalized levels of the selected antibody isotypes that are reactive against the same cN1A, cN1B or fragment thereof in a biological sample of a healthy subject not having IBM, wherein the normalization is performed against a level of albumin in the biological sample of a healthy subject not having IBM, wherein the biological samples are the same for both the healthy subject and subject in need of diagnosis.

In one embodiment of any one of the assays described, the healthy subject does not having IBM, and/or an inflammatory myopathy, and/or a muscle disease.

In one embodiment of any one of the assays described, the detectable presences are determined by the detection limit of the measuring method used in step b.

In one embodiment of any one of the assays described, the measuring method comprises the steps of (a) contacting the biological sample (e.g. blood sample) from the subject with a cN1A, cN1B or protein fragment thereof; (b) forming an antibody-protein complex between the antibody isotype present in the biological sample with the cN1A, cN1B or protein fragment thereof; (c) washing to remove any unbound antibody isotypes; (d) adding at least two detection antibodies that are labeled and are respectively reactive to the at least two antibody isotypes selected for the assay from the biological sample of the subject in order to detect the antibody-protein complex formed in step b; (e) washing to remove any unbound labeled detection antibodies; and (f) converting the label of the at least two detection antibodies to detectable signals, wherein a detectable signal for each of the at least two detection antibodies indicates the presence or level of respective anti-cN1A or anti-cN1B autoantibody isotype in the biological sample of the subject.

In one embodiment of any one of the assays described herein, the detection antibody is labeled by covalently linking to an enzyme, label with a fluorescent compound or metal, or label with a chemiluminescent compound.

In one embodiment of any one of the assays described herein, the cN1B is selected from any one of the known isoforms of NT5C1B described herein.

In one embodiment of any one of the assays described herein, the protein fragment of cN1A or cN1B comprises at least 6 contiguous amino acid residues and is not a full-length cN1A, a full-length cN1B polypeptide or isoforms thereof.

In one embodiment of any one of the assays described herein, the protein fragment of NT5C1A or NT5C1B is an isolated peptide selected from the group consisting of

(SEQ. ID. NO: 1)
AKIFYDNLAPKKKPKSPKPQNAVTIAVSSRALFRMD,
(SEQ. ID. NO: 2)
RALFRMDEEQQIYTEQGVEEYVRYQLEHENEPFSPG,
(SEQ. ID. NO: 3)
SQLRVAFDGDAVLFSDESERIVKAHGLDRFFEHEKA,
(SEQ. ID. NO: 4)
KIRPHIFFDDQMFHVAGAQEMGTVAAHVPYGVAQTP,
(SEQ. ID. NO: 5)
HVAGAQEMGTVAAHVPYGVAQTPRRTAPAKQAPSAQ,
(SEQ. ID. NO: 6)
SQWSRISRSPSTKAPSIDEPRSRNTSAKLPSSSTSS,
(SEQ. ID. NO: 7)
DGDAVLFSDESEHFTKEHGLDKFFQYDTLCESKPLA,
(SEQ. ID. NO: 8)
DGDAVLFSDESERIVKAHGLDRFF,
(SEQ. ID. NO: 9)
DGDAVLFSDESEHFTKEHGLDKFF,
(SEQ. ID. NO: 10)
GDAVLFSDESE,
and
(SEQ. ID. NO: 11)
HGLD(R/K)FF.

In one embodiment of any of the assays described herein, the protein fragment of cN1A or cN1B is fused or conjugated to a heterologous protein (i.e., a non-cN1A or cN1B protein) to form a fusion or chimeric protein such that the fusion protein is not a full-length cN1A, a full-length cN1B polypeptide or isoforms thereof. In some embodiments, the protein fragment of cN1A or cN1B is fused to a polymer.

In one embodiment of any one of the assays described, the measurement method is an immunoassay.

In one embodiment of any one of the assays described, the immunoassay is an enzyme-linked immunosorbent assay.

In one embodiment of any one of the assays described, the immunoassay is an automated immunoassay.

In one embodiment of any one of the assays described, the measuring is performed with the use of a mass spectrometer.

In one embodiment of any one of the assays described, the assay is performed by way of a point-of-care testing (POCT).

In one embodiment of any one of the assays described, the assay is performed by a dipstick, a test strip, a microfluidic chip, a multi-well plate or a cartridge.

In one embodiment of any one of the assays described, the cN1A, cN1B or the isoform thereof, or the peptide fragment thereof, or the isolated peptide, or the fusion protein used is deposited or immobilized on a solid support.

In one embodiment of any one of the assays described, the solid support is in the format of a dipstick, a test strip, a latex bead, a microsphere, a multi-well plate or a cartridge.

In one embodiment of any one of the assay described, the assay further comprises selecting a subject suspected of having IBM.

In another embodiment of any one of the assays described, the assay further comprises selecting a subject in need of diagnosing whether the subject has IBM.

In another embodiment of any one of the assays described, the assay further comprises selecting a subject who is in need of diagnosing whether the subject has IBM and has tested negative for anti-cN1A or anti-cN1B autoantibody isotype IgG.

In one embodiment of any one of the assays described, the assay further comprises selecting the subject for treatment of IBM without having to perform an electromyogram and/or a muscle biopsy on the subject when there is detectable presence or the level of the at least two antibody isotypes selected in the measuring step.

In one embodiment of any one of the assays described, the assay further comprises selecting the subject for treatment of IBM without having to perform an electromyogram and/or a muscle biopsy on the subject when the level of the at least two isotypes selected in step b are at least 5% over that of respective antibody isotype reference levels.

In one embodiment of any one of the assays described, when several biological samples are taken from the subject for the assay, the second time point is selected from the group consisting of at least one month after the start of treatment, at least two months after the start of treatment, at least three months after the start of treatment, at least one month after the first time point, at least two months after the first time point, at least three months after the first time point, at least four months after the first time point, at least half a year after the first time point, one month after the start of treatment, two months after the start of treatment, three months after the start of treatment, one month after the first time point, two months after the first time point, three months after the first time point, four months after the first time point, and half a year after the first time point.

In one embodiment of any one of the assays described, the detectable presences of the at least two antibody isotypes in the second time point indicate the likelihood of continued IBM in the subject.

In one embodiment of any one of the assays described, the assay further comprises comparing the levels of the at least two antibody isotypes in the second time point with the first time points, wherein a decrease in the level of the antibody isotypes taken at the second time point compared to the first time point indicates that the treatment is effective, wherein an increase in the level of the antibodies taken at the second time point compared to the first time point or the levels are approximately the same indicates that the treatment is not effective, wherein when the level of antibodies in the second time point decreases to below a detection limit indicates that there is remission, and wherein the both biological samples of the first and second time points are the same.

In one embodiment, provided herein is a kit comprising a CN1A or CN1B or a protein fragment thereof, or an isolated peptide of CN1A or CN1B, or a fusion protein of CN1A or CN1B; and at least two different detection antibodies, wherein the detection antibody is specific for the antibodies of a patient and specific for at least two of antibody isotypes selected from the group selected from IgG, IgM, IgA, IgD, and IgE.

In one embodiment of the kit, the detection antibodies are detectably labeled.

In one embodiment of the kit, the kit further comprises at least an agent for producing a detectable signal from the detection antibodies.

In one embodiment, provided herein is a system comprising: a measuring module measuring autoantibody isotype information comprising at least two detectable signal from an immunoassay indicating the presence or level of autoantibody isotype that are reactive to at least a CN1A or CN1B or isoform thereof, peptide fragment thereof, the autoantibody isotypes are from a biological sample obtained from a patient, wherein the antibody isotypes are selected from the group selected from IgG, IgM, IgA, IgD, and IgE, and wherein each signal level correspond to an antibody isotype; a storage module configured to store data output from the measuring module; a comparison module adapted to compare the data stored on the storage module with at least two reference levels, and to provide a retrieved content, wherein reference levels are the reference levels for the respective antibodies isotypes measured and are selected from the group selected from IgG, IgM, IgA, IgD, and IgE; and an output module for displaying the retrieved content for the user, wherein the retrieved content, the presence of detectable presence or amount of at least autoantibody isotype reactive against the protein or peptide or fusion protein used of step a, indicates that the patient has IBM or has a relapse of IBM.

In one embodiment of the system, the reference levels comprise data from a population of non-IBM healthy individuals. In another embodiment of the system, the reference levels comprise data from a population of non-IBM individuals who have other myopathies. In another embodiment of the system, the reference levels comprise data from a population of non-IBM healthy individuals and a population of non-IBM individuals who have other myopathies.

In one embodiment, provided herein is a system for evaluating the efficacy of a treatment in a patient with IBM or to facilitate the prognosis evaluation of IBM in a patient, comprising: a measuring module configured to receive and output autoantibody isotype information from a biological sample obtained from a patient, wherein the autoantibody isotype information measures the level of auto antibodies isotypes that are reactive to a CN1A or CN1B or a protein fragment thereof, or an isolated peptide, or a fusion protein described herein, wherein the antibodies isotypes are selected from the group selected from IgG, IgM, IgA, IgD, and IgE; a storage module configured to store output information from the measuring module; a comparison module adapted to compare the data stored on the storage module with a reference data, and to provide a comparison content, wherein the reference data comprises previous data from the same patient wherein the previous data had indicated detectable amounts of autoantibody isotype, and an output module for displaying the comparison content for the user, wherein if there is no detectable amount of autoantibody isotype reactive against the protein or peptide or fusion protein of step a, then the patient is in remission or if there is a reduction of at least 5% compared to a prior reading, then the treatment is effective in the patient.

In one embodiment, provided herein is an assay comprising (a) providing a biological sample from a subject in need of diagnosing, and (b) measuring for a detectable presence or an absence or a level of at least two antibody isotypes that are reactive against a cytosolic 5′-nucleotidase 1A protein (cN1A) or a protein fragment thereof, or a cytosolic 5′-nucleotidase 1B (cN1B) protein or isoforms thereof or a protein fragment thereof in a subject wherein the antibodies isotypes are selected from the group selected from IgG, IgM, IgA, IgD, and IgE, wherein the measuring method comprises the steps of (i) contacting the blood sample from the subject with a cN1A, cN1B or protein fragment thereof; (ii) forming an antibody-protein complex between the antibody isotype present in the blood sample with the cN1A, cN1B or protein fragment thereof; (iii) washing to remove any unbound antibody; (iv) adding at least two detection antibodies that are labeled and are respectively reactive to the at least two antibody isotypes selected for the assay from the blood sample of the subject in order to detect the antibody-protein complex formed in measuring step b; (v) washing to remove any unbound labeled detection antibodies; and (vi) converting the label of the at least two detection antibodies to detectable signals, wherein the a detectable signal for each of the at least two detection antibodies indicates the presence or level of respective anti-cN1A or anti-cN1B autoantibody isotype in the biological sample of the subject.

In one embodiment, provided herein is a method of treatment of inclusion body myositis (IBM) in a subject in need thereof comprising performing an assay described herein, selecting the subject for treatment if there is a detectable signal for each of the at least two detection antibody isotype selected for the assay; and administering an immunosuppressive therapy to the subject.

In one embodiment, provided herein is a method of treatment of inclusion body myositis (IBM) in a subject in need thereof comprising performing a diagnosis of IBM using any one of the kits or systems of described herein, selecting the subject for treatment if there is a detectable signal for each of the at least two detection antibody isotype selected for the assay; and administering an immunosuppressive therapy to the subject.

In one embodiment, provided herein is a method of treatment of inclusion body myositis (IBM) in a subject in need thereof comprising diagnosing whether the subject has IBM by measuring for a detectable presence or an absence or a level of at least two antibody isotypes that are reactive against a cN1A or a protein fragment thereof, or a cN1B protein or isoforms thereof or a protein fragment thereof in a biological sample from the subject, wherein the antibody isotypes are selected from the group selected from IgG, IgM, IgA, IgD, and IgE, and administering an immunosuppressive therapy to the subject when there is a detectable presence of two antibody isotypes selected or at least 5% above the reference level for the two antibody isotypes selected.

In one embodiment of the treatment method, the measuring for a detectable presence or an absence or a level of at least two antibody isotypes that are reactive against a cN1A or a protein fragment thereof, or a cN1B protein or isoforms thereof comprises the following steps: (i) contacting the blood sample from the subject with a cN1A, cN1B or protein fragment thereof; (ii) forming an antibody-protein complex between the antibody isotype present in the blood sample with the cN1A, cN1B or protein fragment thereof; (iii) washing to remove any unbound antibody; (iv) adding at least two detection antibodies that are labeled and are respectively reactive to the at least two antibody isotypes selected for the assay from the blood sample of the subject in order to detect the antibody-protein complex formed in measuring step b; (v) washing to remove any unbound labeled detection antibodies; and (vi) converting the label of the at least two detection antibodies to detectable signals, wherein the a detectable signal for each of the at least two detection antibodies indicates the presence or level of respective anti-cN1A or anti-cN1B autoantibody isotype in the biological sample of the subject.

Also provided herein, in some aspects, are methods of treatment of inclusion body myositis (IBM) in a subject in need thereof, the methods comprising administering an immunosuppressive therapy to a subject having a detectable presence or a level of at least two antibody isotypes that are reactive against a cytosolic 5′-nucleotidase 1A protein (cN1A) or a protein fragment thereof, or a cytosolic 5′-nucleotidase 1B (cN1B) protein or isoforms thereof or a protein fragment thereof, wherein the antibodies isotypes are selected from the group selected from IgG, IgM, IgA, IgD, and IgE.

In some embodiments of these aspects and all such aspects described herein, the methods further comprise comparing the levels of the at least two antibody isotypes at a second time point, wherein a decrease in the level of the antibody isotypes taken at the second time point indicates that the therapy is effective, wherein an increase in the level of the antibodies taken at the second time point are approximately the same indicates that the treatment is not effective, and wherein when the level of antibodies in the second time point decreases to below a detection limit indicates that the subject is in remission.

### DEFINITIONS OF TERMS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs.

As used in this specification and the appended claims, the singular forms “a,” “an,” and “the” include plural references unless the context clearly dictates otherwise. Thus for example, references to “the assay” includes one or more assays, and/or steps of the type described herein and/or which will become apparent to those persons skilled in the art upon reading this disclosure and so forth. Similarly, the word “or” is intended to include “and” unless the context clearly indicates otherwise. Although methods, assays and materials similar or equivalent to those described herein can be used in the practice or testing of this disclosure, suitable methods, assays and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%.

The term “protein fragment” of a cN1A or a cN1B refers to any subject polypeptide or protein having an amino acid residue sequence shorter than that of a polypeptide whose amino acid residue sequence is described herein. A fragment of a cN1A or a cN1B is a shortened or truncated cN1A or cN1B proteins. The polypeptide can have N-terminus or C-terminus truncations and/or also internal deletions. In one embodiment, a protein fragment of a cN1A or a cN1B have about 50 or less contiguous amino acid residues. In some embodiments, protein fragments of a cN1A or a cN1B have about 40, 30 or 20 contiguous amino acid residues. In one embodiment, protein fragments of a CN1A or a CN1B have about 10 or less than contiguous amino acid residues. In one embodiment, the “protein fragment” of a CN1A or a CN1B is one that the autoantibody isotypes from a biological sample, e.g. a blood sample, of a patient having IBM reacts with, in other words, the “protein fragment” of a CN1A or a CN1B in an antigen of the autoantibody isotypes from a biological sample of a patient having IBM. In one embodiment, the “protein fragment” of a CN1A or a CN1B is a peptide.

In one embodiment, a peptide of CN1A or a CN1B as used herein refer to a polymer of 50 or less contiguous amino acid residues derived from a full-length CN1A or a full-length CN1B. In one embodiment, “peptide” and “protein fragment” are used interchangeably.

As used herein, the term “treat′ or treatment” refers to reducing or alleviating at least one adverse effect or symptom associated with IBM. These include reducing the amount of autoantibody isotypes against a CN1A, a CN1B protein, reducing, inhibiting or stopping the production of auto-antibody isotypes against such proteins, suppression of the immune system, and reducing the inflammation and degradation/damage associated with the activities of the autoantibodies, progression of muscle weakness, muscle pain and fatigue. In one embodiment, treatment administered to the patient encompassed more than type or kind of treatment strategy or approach or target. For example, the treatment is a combination of immunosuppression by drug administration, a physical exercise therapy, a nutrition program and a gene or biologic therapy.

The term “subject” as used herein refers to a mammal. In another embodiment, the subject is a human. As used herein, the terms “subjects”, “individuals” or “patients” are used interchangeably. A subject can be male or female.

The term “consisting of” refers to assays, methods, devices, kits and systems respective components thereof as described herein, which are exclusive of any element not recited in that description of the embodiment.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of elements that do not materially affect the basic and novel or functional characteristic(s) of that embodiment.

As used herein, the term “idiopathic IBM” is currently used to describe IBM that is not caused by any known etiology. In one embodiment, “idiopathic IBM” is not PM or DM.

As used herein, the term “autoantibodies” and “antibodies” against a CN1A or a CN1B are used interchangeably. Such antibodies are produced by a subject and in reactive to an endogenous protein in the subject. In one embodiment, the “autoantibodies” are antibodies reactive against a self-protein. In one embodiment, the “autoantibodies” and “antibodies” against a CN1A or a CN1B also encompassed all the antibody isotypes, i.e., isotypes IgA, IgM, IgD, IgG, and IgE. The IgA isotypes includes IgA1 and IgA2, and the IgG isotypes includes IgG1, IgG2a, IgG2b, IgG3 and IgG4.

In one embodiment, the term “antibody isotype” is used interchangeably with the term “antibody subtype.”

As used herein, the term “endogenous protein” is a protein encoded by the genome of the subject. In one embodiment, an “endogenous protein” is a self-protein with respect to the subject.

As used herein, the term “reactive” when used in the context of an antibody or an antibody isotype refers to the binding of the antibody or antibody isotype to its antigen. For example, an anti-CN1A antibody is reactive to CN1A, the antibody's antigen. In one embodiment, an antibody is “reactive” to an antigen also means the antibody is targeted against that antigen.

The term “identity” here refers to the degree of relatedness between two or more protein sequences, which is determined by the match between these sequences. The percentage identity is obtained as the percentage of identical amino acids in two or more sequences taking account of gaps and other sequence features.

The terms “identical” or percent “identity”, in the context of two or more polypeptide or protein sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.”

As used herein, the term “fusion protein” or “fusion polypeptide” refers to a protein created by joining two genes or two proteins/peptides together. In the laboratory, this can be achieved through the creation of a fusion gene which is done through the removal of the stop codon from a DNA sequence of the first protein and then attaching the DNA sequence of the second protein in frame. The resulting DNA sequence will then be expressed by a cell as a single protein. In a fusion protein, the two proteins that will be joined together with a linker or spacer peptide added between the two proteins. This linker or spacer peptide often contain protease cleavage site to facilitate the separation of the two proteins after expression and purification The making of fusion protein as a technique is commonly used for the identification and purification of proteins through the fusion of a GST protein, FLAG peptide or a hexa-his peptide.

By “conjugated” is meant the covalent linkage of at least two molecules. As described herein, a CN1A or CN1B peptide is conjugated to a polymer or a solid support, a label (e.g., a latex bead or gold particles) or to a non-related (i.e., non-CN1A or non-CN1B peptide or protein).

As used herein, the term “heterologous peptide or polypeptide” refers the polymer of amino acid residue that are not naturally associated or are part of the CN1A or CN1B protein as encoded by the genome.

In one embodiment, the term “statistically significant” or “significantly” refers to statistical significance and generally means a two standard deviation (2SD) difference, above or below a reference value, usually the average or the median. In one embodiment, “statistically significant” or “significantly” refers to one SD above or below a reference value, e.g. the average. In one embodiment, “statistically significant” or “significantly” refers to three SD above or below a reference value, e.g. the average. In one embodiment, “statistically significant” or “significantly” refers to four SD above or below a reference value, e.g. the average. Additional definitions are provided in the text of individual sections below.

The terms “increased” or “increase” means an increase of at least 5% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease,” “reduce,” “reduced”, and “reduction” are all used herein generally to mean a decrease by at least 5% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment. In one embodiment, a “decrease” is the absence of autoantibodies as measured by the detection limit of an assay method for antibodies, e.g., ELISA or Western blot analysis.

In one embodiment, as used herein, the “detection limit” of an assay method for antibodies is the background level of the signal produced by the measuring method.

In one embodiment, as used herein, an “effective” treatment for IBM is evaluated by a reduction of at least 5% of autoantibody isotypes that are reactive to cN1A or cN1B or isoforms thereof or protein fragments therefrom, the autoantibody isotypes are found in a biological sample (e.g., a blood sample) of a patient, the reduction is compared to a prior reading of autoantibody isotypes in the biological sample of the same patient or to a reference level of such autoantibody isotypes in the biological sample(s) of healthy subjects not having IBM. In other embodiments, the reduction can be at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of such autoantibody isotypes detectable in the biological sample of the patient. The prior reading was obtained when the patient was having IBM, i.e., that IBM was active in the patient and the prior reading was above that found in non-IBM patients or healthy non-IBM patients.

In another embodiment, as used herein, an “effective” treatment for IBM is evaluated by the non-detectable presences of autoantibody isotypes that are reactive to cN1A or cN1B or isoforms thereof or protein fragments therefrom, the autoantibody isotypes are being analyzed from in a biological sample (e.g., a blood sample) of a patient.

In one embodiment, the non-detectable presences of autoantibody isotypes means below detectable limit of the autoantibody isotypes being tested.

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest.

The term “labeled antibody”, as used herein, includes antibodies that are labeled by a detectable means and include, but are not limited to, antibodies that are enzymatically, radioactively, fluorescently, colorimetrically and chemiluminescently labeled. Antibodies can also be labeled with a detectable tag, such as c-Myc, HA, VSV-G, HSV, FLAG, V5, or HIS. The detection and quantification of an autoantibody present in a blood sample correlate to the intensity of the signal emitted from the detectably labeled antibody.

The term “label” refers to a composition capable of producing a detectable signal indicative of the presence of the target autoantibody present in a biological sample, e.g., a blood sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “agent” as used herein refers to a chemical entity or biological product, or combination of chemical entities or biological products that are used in developing the detectable signal indicative of the presence of the target autoantibody present in a blood sample.

The term “support” refers to conventional supports such as beads, particles, dipsticks, fibers, filters, membranes and silane or silicate supports such as glass slides.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” can refer to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

As used herein, the term “sensitivity” in the context of the assays described refers to a measure of the proportion of actual positives which are correctly identified as such e.g. the percentage of sick people who are correctly identified as having the condition. The sensitivity of a test is the proportion of people that are known to have the disease who test positive for it. Sensitivity relates to the test's ability to identify positive results. “Sensitivity” is also called the true positive rate. A test with high sensitivity can be considered as a reliable indicator when its result is negative, since it rarely misses true positives among those who are actually positive. For example, a sensitivity of 100% means that the test recognizes all actual positives—i.e. all sick people are recognized as being ill. In one embodiment, sensitivity in calculated by the following formula: Sensitivity=number of positive IBM/number of total IBM samples

As used herein, the term “specificity” in the context of the assays described refers to a measure of the proportion of negatives which are correctly identified as such (e.g. the percentage of healthy people who are correctly identified as not having the condition, sometimes called the true negative rate). The specificity of a test is defined as the proportion of patients that are known not to have the disease who will test negative for it. Specificity relates to the test's ability to identify negative results. Highly specific tests rarely miss negative outcomes, so they can be considered reliable when their result is positive. Therefore, a positive result from a test with high specificity means a high probability of the presence of disease. In one embodiment, specificity in calculated by the following formula: Specificity=number of negative non-IBM/number of total non-IBM samples

In one embodiment, the term “non-invasive” in the context of the described diagnosis test/assay means not having to perform a muscle tissue biopsy.

### BRIEF DESCRIPTION OF THE DRAWINGS

FIG. 1A. Western blots of control experiments demonstrating the specificity of secondary antibody reagents. Anti-IgM, anti-IgA, and anti-IgG secondary antibodies react against only IgM heavy chain (˜70 kDa), IgG heavy chain (˜50 kDa) and IgA heavy chain (˜50 kDa) respectively.

FIG. 1B. Western blots demonstrating the presence of human serum IgM, human serum IgA, and human serum IgG anti-43 kDa autoantibodies reacting to a 43 kDa protein in skeletal muscle lysates. Preabsorbtion (blocking) of the human serum were performed by incubation with recombinant cytosolic 5′-nucleotidase 1A protein (NT5C1A or cN1A) prior to blotting demonstrates loss of the 43-kDa band reactivity, confirming that the 43 kDa skeletal muscle protein to which these autoantibodies react is cN1A.

FIG. 1C. Western blots showing that preabsorbtion of human serum by incubation with recombinant cN1A in solution results in loss of Western blot reactivity against denatured cN1A, demonstrating that serum IgM, IgA, and IgG autoantibodies react in solution to recombinant non-denatured cN1A.

FIG. 1D. Patterns of Western blot reactivity of various patients anti-cN1A autoantibodies to recombinant cN1A. Patient 1 has strong IgG and IgM but weak IgA autoantibody reactivity; patient 2 has strong IgG with no IgM and weak IgA reactivity; and patient 3 has strong reactivity for all Ig substypes.

FIG. 2A. Graphs showing the ELISA performances for detection of IgM, IgA, and IgG autoantibodies in 205 patients (49 with IBM, 156 without IBM). Reproducibility of ELISAs, with high linear correlations in duplicate assays.

FIG. 2B. Distributions of ELISA absorbance signals detecting each autoantibody subtype in IBM (N=49) and other (N=156) samples.

FIG. 2C. Diagnostic performance of single thresholds for each ELISA, assessed by receiver operator characteristic (ROC) analyses. AUC=area under the curve.

FIGS. 3A-3C. Scatterplot of pairwise autoantibody subtypes demonstrate some patients have high-levels of one subtype but not another. The highest correlation was between IgM and IgA autoantibodies. The variable subtype responses indicate diagnostic utility to measuring multiple subtypes.

FIG. 3A. Scatterplot of pairwise autoantibody subtypes IgG vs IgM.

FIG. 3B. Scatterplot of pairwise autoantibody subtypes IgG vs IgA.

FIG. 3C. Scatterplot of pairwise autoantibody subtypes IgM vs IgA.

FIGS. 4A and 4B. Classification tree for the determination of IBM diagnosis.

FIG. 4A. Principal components analysis showing most variance is accounted by the first component (IgM autoantibody level).

FIG. 4B. A classification tree built from IgM, IgA, and IgG autoantibody levels of 205 patients. Nodes shown in orange and pie charts with blue IBM and green not-IBM. The topmost central node, for example, indicates that 23.9% of the 205 patients have IBM. An IgM level of >1.685 (left branch) classifies 36 patients of which 77.8% have IBM, while an IgM level <1.685 (right branch) classifies 169 patients, of which 12.4% have IBM. Further classifications performed with IgG and IgA levels results in final classifications (terminal nodes) with sensitivity of 78% and specificity of 92%.

FIGS. 5A (top view) and 5B (side view) are schematic diagrams of an exemplary test strip assay for determining the presence and/or level of autoantibody isotype IgG and IgM in a fluid biological sample.

FIG. 6 is a schematic diagram showing the interpretation of the results obtained using the test strip shown in FIG. 5.

FIG. 7 is a block diagram showing an exemplary system for IBM diagnosis comprising analyzing the presence or level of autoantibody isotypes reactive against cN1A or cN1B.

FIG. 8 is an exemplary set of instructions on a computer readable storage medium for use with the systems described herein.

### DETAILED DESCRIPTION

Embodiments and all aspects of the technology described herein are based on the discovery that in addition to the previously recognized IgG autoantibodies against the skeletal muscle protein, cytoplasmic 5′-Nucleotidase 1A (NT5C1A or cN1A), IBM patients have circulating IgM anti-cN1A and IgA anti-cN1A autoantibodies. The presence of IgM and IgA anti-cN1A antibody isotypes have been identified in IBM patients, in addition to the known presence of IgG anti-cN1A antibody isotype. Additionally, ELISA assays for all 3 antibody isotypes have been developed, in addition to the previously reported western blot,⁴ dot blot,⁵ and immunoprecipitation assays.⁶

Moreover, there are differing patterns of these autoantibody isotypes present among patients. The inventors demonstrated that patients with IBM can have differing patterns of anti-cN1A autoantibody isotypes. Measurement of IgA and IgM anti-cN1A antibody isotypes detects some patients in whom IgG autoantibodies were not detected.

The inventors found that ELISA assays for measurement of all 3 subtypes autoantibodies against cN1A can be perform easily with high reproducibility. Plasma and serum samples from 205 patients (49 with IBM, 156 without IBM) were studied for the presence of IgM, IgA, and IgG autoantibodies using immunoblots and ELISAs. The inventors also used computational machine learning approaches to learn and classify diagnosis strength base on single antibody isotype analysis or combination antibody subtypes analyses. Healthy subjects free of any inflammation conditions and patients with other myositis, e.g., PM and DM, do not have detectable autoantibodies that react, bind or target CN1A or CN1B.

CN1A or CN1B are naturally occurring proteins in a patient and are considered to be self-proteins with respect to that patient. The function of immune system in a patient is to protect the patient from foreign pathogens. In order to perform this function, the immune system must be able to distinguish self-proteins from foreign proteins; the cells of the immune system should not bind self-proteins and should only bind to foreign proteins from pathogens. Therefore, in a normal healthy patient, there should not be any antibody isotypes reactive to CN1A or CN1B. An antibody that targets, binds, and is reactive against a self-protein is an autoantibody.

Since the presence of anti-CN1A and/or anti-CN1B autoantibodies is found in IBM patients and not in healthy subjects or patients with other myositis, detection of anti-NT5C1A and/or anti-NT5CN1B autoantibodies can be used to diagnose IBM, thereby providing an easily non-invasive method of diagnosing IBM in lieu of the invasive method of a muscle biopsy.

Accordingly, a simple blood sample can be used to test for and detect autoantibody isotype reactive against a CN1A, or CN1B, or protein fragments thereof. Such a method would be highly favorable over the current diagnostic method of a muscle tissue biopsy which is an invasive technique. In one embodiment, the autoantibodies can be detected by an immunoassay wherein an antibody-protein complex is formed. In one embodiment, the immunoassay is a serological immunoassay.

The inventors discovered that single isotype autoantibody analysis for IgM, IgA, and IgG anti-cN1A autoantibodies were detected with similar sensitivities (37-59%) and specificities (90-99%) depending on threshold values used, and that IBM patients had differing patterns of autoantibody presence. More importantly, combination assays including measurements of three autoantibody levels improved the optimal 51% diagnostic sensitivity from using the IgG subtype alone to 73%, with minor reduction in specificity from 94% to 92%. These differing patterns of antibody isotypes allow for improvements in diagnostic approaches by consideration of levels of more than one antibody isotype, e.g., three autoantibody isotypes, with resulting sensitivity and specificity of 73%/92%. Because IBM has a high rate of misdiagnosis (54%) and delayed diagnosis (5.2 years)′ and diagnosis generally includes muscle biopsy, the improvements in diagnostic performance of blood testing may translate to better care and treatment for patients with IBM. Therefore, diagnostic assays involving the measurement of more than one anti-cN1A antibody isotypes, e.g., using all 3 autoantibody subtypes, offer higher diagnostic performance, i.e., diagnostic sensitivity, than those measuring the IgG isotype alone. Here, the threshold values are the average level of the respective anti-cN1a autoantibody isotypes found a population of humans not having positively been diagnosed with IBM. In some embodiments, the threshold values are the average level of the respective anti-cN1a autoantibody isotypes found a population of health non-IBM humans, or are the average level of the respective anti-cN1a autoantibody isotypes found a population of humans having other inflammatory myopathies but not IBM, e.g., DM, PM, myasthenia gravis, necrotizing myopathy, and/or polymyositis. In one embodiment, the threshold values are the average level of the respective anti-cN1a autoantibody isotypes found a population of health non-IBM humans and humans having other inflammatory myopathies but not IBM. The terms “threshold values” and “references levels” are used interchangeably.

It should be noted that the current serological diagnostic tests for PM and DM, e.g., anti-Jo antibodies, have a sensitivity of only about ˜20% and a specificity of less than 50%. This means that the current serological tests for PM and DM under diagnose about 80% of the time. Accordingly, compared to the serological diagnostics available for PM and DM, or the current IBM diagnostic assay comprising only measuring IgG autoantibodies, the present non-invasive diagnostic assays described herein comprising measuring a combination of more than one autoantibody isotype for IBM diagnosis have greater sensitivity and specificity at detecting IBM.

Accordingly, in one embodiment, provided herein is a assay for diagnosing the likelihood of IBM, the assay comprises (a) providing a biological sample from a subject in need of diagnosing, and (b) measuring for a detectable presence or an absence or a level of at least two antibody isotypes that are reactive against a cN1A or a protein fragment thereof, or a cytosolic 5′-nucleotidase 1B (cN1B) protein or isoforms thereof or a protein fragment thereof for diagnosing or detecting IBM in a subject wherein the antibody isotypes are selected from the group selected from IgG, IgM, IgA, IgD, and IgE. In another embodiment, the antibody isotypes are selected from the group selected from IgG1, IgG2a, IgG2b, IgG3, IgG4, IgM, IgA1, IgA2, IgD, and IgE. In one embodiment, the biological sample is a blood. In one embodiment, a detectable presence of the at least two isotypes of anti-cN1A autoantibodies indicates the likelihood of IBM in the patient.

The non-invasive diagnostic methods and assays that have greater sensitivity and with no loss of specificity rely on detecting the absence/presence and/or measuring the level of more than one autoantibody isotypes that is against cN1A or cN1B. In other words, several antibody isotypes are studied simultaneously in order to make the determination of the likelihood of IBM in an individual, e.g., a combination of anti-cN1A IgG, anti-cN1A IgA, anti-cN1A IgM, anti-cN1A IgD and anti-cN1A IgE antibody isotypes are studied simultaneously. Studying more than one autoantibody isotypes gives the non-invasive diagnostic assays better sensitivity of detecting IBM.

This diagnostic assay is an improved, non-invasive diagnostic assay over that currently known in the art. In one embodiment, the improvement is that the assay has increased diagnostic sensitivity with high specificity. This can be adapted for use in prognosis evaluation of a patient being treated for IBM, for monitoring IBM recurrence in patients who have been successfully treated for IBM and the IBM is in remission, and also can be useful for evaluating the effectiveness of a treatment or a therapeutic drug that is being used on a patient with IBM. In other words, the assays described herein can be used to (1) verify whether myositis, muscle weakness and joint pain in a patient is due to autoantibody isotypes immunoreactive to CN1A and/or CN1B; (2) confirm a suspected case of IBM before other symptoms occur or progress further; often IBM diagnosis occurs after many year of misdiagnosis; (3) determine whether symptoms of muscle weakness are caused by a known autoimmune disease or disorder, a muscle or a nerve problem; (4) differentiate between some types of myositis and myopathies such as PM, DM, dystrophies, and congenital myopathies; (5) follow the course (progression, remission, relapse or status quo) of IBM myositis in a patient during a treatment program; and (6) permit evaluation of the efficacy of a treatment for IBM.

Traditionally, IBM patients were definitively diagnosed with IBM by muscle tissue biopsies; their muscles have the classic appearance of vacuole-like holes in the muscles, deposits of abnormal proteins within the cells and presence of filamentous inclusions in the muscles. Alternatively, these patients were differential diagnosed whereby all other known possible causes of symptoms associated with IBM have been systematically ruled out.

Inclusion body myositis (IBM) is a poorly understood autoimmune and degenerative disease of muscle. Although T-cell mediated autoimmunity has been recognized for 30 years as a central component of IBM, the identification of an IBM autoantigen has remained a mystery until recently. The recognition of IBM B-cell autoimmunity within the last 10 years¹⁻³ resulted in the identification of circulating autoantibodies against an approximately 43 kDa muscle IBM autoantigen (anti-IBM43) two years ago.⁴ A method of detecting these anti-IBM43 autoantibodies was demonstrated to be a highly sensitive and specific blood diagnostic test for IBM.⁴ Recent studies have identified the autoantigen to which anti-IBM43 autoantibodies are directed as cytoplasmic 5′-nucleotidase 1A (cN1A; NT5C1A)^{5,6} and found that cN1A is abnormally distributed in IBM muscle, localizing to areas of myonuclear degeneration and rimmed vacuoles.⁵ These later findings provide a mechanistic link between the degenerative and immune features of IBM.⁵ See International PCT application No: PCT/US2012/37275 and PCT/NL2012/050482 which is hereby incorporated by reference in its entirety.

Because IBM has an estimated approximately 54% rate of misdiagnosis with mean delay to diagnosis of 5.2 years,⁷ and the diagnosis usually includes muscle biopsy, a non-invasive diagnostic test with high sensitivity for IBM would be of benefit to patients. The previously described serological method in which these anti-cN1A autoantibodies are captured from blood, and then visualized as bands on nitrocellulose membranes containing skeletal muscle lysates, is of moderate diagnostic sensitivity and high diagnostic specificity.⁴ A variety of approaches are possible for trapping and visualizing these autoantibodies, including the use of purified antigen (recombinant cN1A).^{5,6}

All previously published methods for detection of anti-IBM43 autoantibodies have used secondary antibodies specifically directed against the IgG subclass for the visualization of autoantibody captured on membranes,⁴⁻⁶ or have used immunoprecipation with protein A which has high affinity for human IgG1 and IgG2 and only moderate affinity for IgA and IgM. Thus, the previously identified autoantibodies are all of the IgG subclass or of undetermined subclass, and the previous reports do not enable one skilled in the art to make a determination whether or not anti-cN1A IgM or anti-cN1A IgA or anti-cN1A IgD or anti-cN1A IgE autoantibodies are also present. In fact, there is no report or evidence of the presence of any other anti-cN1A antibody isotypes associated with IBM beside the IgG isotype.

Here, the presence of both IgM and IgA anti-cN1A autoantibody subtypes are demonstrated, as well as ELISA-based methods for the detection of IgM, IgA, and IgG, and the use of diagnostic algorithms for optimizing IBM serological diagnosis based on analyses of three immunoglobulin subtypes. Since the detectable presence of autoantibodies targeting cN1A/cN1B is found in IBM patients and not in healthy subjects or patients with other non-IBM myositis or myopathies, detection of such autoantibodies can be useful for diagnosing, detecting and/or confirming IBM in a subject, especially when there is currently no approved, easy, non-invasive method of diagnosing IBM except via the invasive method of a muscle biopsy.

In another embodiment, provided herein is an assay for evaluating the efficacy of a treatment for a patient having IBM, the assay comprising (a) measuring for an absence or a presence or a level of at least two antibody isotypes that are reactive against a cN1A, or a cN1B protein or isoforms thereof in at least two biological samples that are chronologically separated apart, wherein the antibodies isotypes are selected from the group selected from IgG, IgM, IgA, IgD, and IgE; (b) comparing the absence/presence or levels of the autoantibody isotypes measured for the at least two biological samples; and (c) evaluating the prognosis of the subject or the treatment efficacy based on the detectable presence or the level of the at least two antibody isotypes selected in above measuring step (a). For example, taking a biological sample at a first time point and a second time point and perhaps several subsequent time points thereafter. By comparing the measured autoantibody isotype results of a later time point with the measured autoantibody isotype results of an earlier time point, usually the most recent earlier time point in chronological time, a determination of prognosis of the subject being treated can be made, or a determination of the effectiveness of the treatment protocol can be made. For example, if there is a decrease in autoantibody isotype in the most recent earlier time point compared to with the measured autoantibody isotype results of an earlier time point; this indicates that the patient is getting better and that the treatment is working. In another embodiment, the antibody isotypes are selected from the group selected from IgG1, IgG2a, IgG2b, IgG3, IgG4, IgM, IgA1, IgA2, IgD, and IgE.

In one embodiment, provided herein is an assay comprising (a) measuring for an absence or a presence or a level of at least two antibody isotypes that are reactive against a cN1A, or a cN1B protein or isoforms thereof in at least two biological samples obtained from a subject at different times for a prognosis evaluation of the subject with IBM or for evaluating the efficacy of a treatment in the subject with IBM, wherein the different times are a first time point and at least a second time point, wherein the second time point is after the first time point, and wherein the antibodies isotypes are selected from the group selected from IgG, IgM, IgA, IgD, and IgE; and (b) evaluating the prognosis of the subject or the treatment efficacy based on the detectable presence or the level of the at least two antibody isotypes selected in above measuring step (a). In another embodiment, the antibody isotypes are selected from the group selected from IgG1, IgG2a, IgG2b, IgG3, IgG4, IgM, IgA1, IgA2, IgD, and IgE.

In one embodiment of any one of the assays described, the patient has IBM and is being treated for IBM. In one embodiment of any one of the assays described, the subject is being treated for IBM. In one embodiment of any one of the assay described, the treatment is experimental. In one embodiment of any one of the assays described, the subject had previously been successfully treated for IBM and the IBM was in remission.

In one embodiment of any one of the assays described, the antibody isotypes are selected from the group selected from IgG1, IgG2a, IgG2b, IgG3, IgG4, IgM, IgA1, IgA2, IgD, and IgE.

Currently, there is no known effective treatment for IBM. In one embodiment, an effective treatment for IBM would at the minimum encompass reducing the level of autoantibody isotypes to a CN1A or CN1B, or a peptide fragment, or an isolated peptide, or a fusion protein described herein thereof. In another embodiment, an effective treatment for IBM would at the minimum encompass reducing at least one symptom known to be associated with IBM, e.g., muscle pain or weakness.

In one embodiment of any one of the assays described, the at least two biological samples that are chronologically separated apart comprises a first biological sample and a second biological sample from the patient wherein the first sample is taken at a first time point and the second sample is taken at a second time point, and the second time point is after the first time point.

In one embodiment of any one of the assays described, when at least two biological samples are taken for the assay, the biological samples are chronologically separated apart. In one embodiment of any one of the assays described, when more than two biological samples are taken for the assay, the samples are chronologically separated apart. In one embodiment of any one of the assays described, the more than two biological samples obtained for the assay comprise periodic biological samples are taken as long as the patient is being treated for IBM and it has not been shown that the IBM is in remission. For example, a patient was diagnosed in January 2013 and began treatment in February 2013. Biological samples are taken during the diagnosis phase, in January 2013 and then subsequently and periodically in February 2013 onwards until the assay indicate below detectable limits of the anti-cN1A and/or anti-cN1B autoantibody isotypes. Periodic sampling can be every month, every two months, every three months, every four months, every five months, every six months, or annually.

In one embodiment of any one of the assays described, when there is no detectable presence (ie., below a detection limit of the measuring method) of the tested autoantibody isotypes taken at the second time point compared to the first time point and where there was a detectable presence of the autoantibody isotypes at the first time point indicates that the treatment is effective and that the patient is in remission. Alternatively, when several subsequent and periodic samples are taken, when the autoantibody isotypes measured at the most recent time point is below the detection limit of the measuring method, this indicates that the treatment is effective and that the patient is in remission.

In alternate embodiment of any one of the assays described, when there is continued detectable presence of the tested autoantibody isotypes taken at the second time point compared to the first time point and where there was a detectable presence of the autoantibody isotypes tested at the first time point indicates that the IBM in the patient is approximately the same, and indicates that the treatment is not effective.

In some embodiments of any one of the assays described, when there is a decrease in the level of the autoantibody isotypes tested taken at the second time point compared to the first time point indicates that the treatment is effective; when there is an increase in the level of the autoantibody isotypes tested taken at the second time point compared to the first time point or the levels are approximately the same indicates that the treatment is not effective; and when the level of autoantibodies in the second time point decreases to below a detection limit indicates that the treatment is effective and the patient is in remission.

In one embodiment of any one of the assays described when several biological samples are taken from the subject for the assay, the first time point is before any treatment for IBM has been administered to the patient. In one embodiment of any one of the assays described when several biological samples are taken from the subject for the assay, the first time point is after a treatment for IBM has been administered to the patient. In another embodiment, the first time point is after the start of at least one treatment. In some embodiments, more than one type of treatment is administered concurrently to the patient.

In one embodiment of any one of the assays described when several biological samples are taken from the subject for the assay, the second time point is after a treatment for IBM has been administered to the patient. For example, the second time point is taken after a period of time after the start of a treatment for IBM has been administered to the patient. The a period of time after the start of a treatment for IBM can be anywhere from one week, one month, two months, three months, four months, five months, half a year or one year after the start of a treatment for IBM. In one embodiment, the second time point is at least one month after the start of at least one treatment.

In another embodiment, the second time point is at least one month after the first time point and both the first time point and the second time point are after the start of at least one treatment. In some embodiments, a third and fourth sample time points are performed. In any embodiment, the comparison is always between the data of a later time point with the data of an earlier time point, the earlier time point can be the immediate prior time point of the later time point.

In one embodiment of any one of the assays described when several biological samples are taken from the subject for the assay, the first time point is before any treatment for IBM has been administered to the patient and the second time point is after a treatment for IBM has been administered to the patient. In one embodiment of any one of the assays described when several biological samples are taken from the subject for the assay, both the first time point and the second time point are after a treatment for IBM has been administered to the patient.

In one embodiment of any one of the assays described when several biological samples are taken from the subject for the assay, the second time point is selected from the group consisting of at least one month after the start of treatment, at least two months after the start of treatment, at least three months after the start of treatment, at least one month after the first time point, at least two months after the first time point, at least three months after the first time point, at least four months after the first time point, at least half a year after the first time point, one month after the start of treatment, two months after the start of treatment, three months after the start of treatment, one month after the first time point, two months after the first time point, three months after the first time point, four months after the first time point, and half a year after the first time point. In one embodiment of any one of the assays described when several biological samples are taken from the subject for the assay, the second time point can be anywhere from one month to a year after the first time point. In one embodiment of any one of the assays described when several biological samples are taken from the subject for the assay, the second time point can be anywhere from one month to a year after a treatment for IBM has been administered to the patient.

In one embodiment of any one of the assays described, the detectable presences of the at least two antibody isotypes in the second time point indicate the likelihood of continued IBM in the subject.

In one embodiment of any one of the assays described, the assay further comprises comparing the levels of the at least two antibody isotypes in the second time point with the first time points, wherein a decrease in the level of the antibody isotypes taken at the second time point compared to the first time point indicates that the treatment is effective, wherein an increase in the level of the antibodies taken at the second time point compared to the first time point or the levels are approximately the same indicates that the treatment is not effective, wherein when the level of antibodies in the second time point decreases to below a detection limit indicates that there is remission, and wherein the both biological samples of the first and second time points are the same.

The subject has initially been evaluated with symptoms consistent with IBM and has detectable amounts of autoantibody isotypes against CN1A or the CN1B, or the peptide fragment thereof, or an isolated peptide, or a fusion protein described herein. Upon treatment, for example, with novel immunosuppressive therapy or other experimental treatment, over time, there is a decrease in the amount of detectable autoantibody. In an ideal case, the amount of autoantibody isotypes should fall below the detectable level of the detection/measuring methods described herein and the subject is deemed to be in remission for IBM. A decrease in the level of autoantibodies in the second time point compared to the first time point (or a later time point compared to an earlier time point) is at least 5%, at least 5%, at least 20%, at least 30%, at least 40%, at least 50%, at least 60%, at least 70%, at least 80%, at least 90%, 100% and all the percentages between 10-100% drop in the titer of autoantibody isotypes. In one embodiment, below the detection limit is when the level of antibody isotypes is reduced to between 95-100% or more compared to the first time point when the subject was initially evaluated with symptoms consistent with IBM, tested positive for autoantibody isotypes, and no treatment has been administered to the subject. The assay methods used for measuring the levels of autoantibody isotypes are identical for all the samples collected at different time points from the subject. Decreasing titers of auto antibodies indicates that the treatment is effective in the subject.

In one embodiment, there is an increase in the level of antibody isotypes in the second time point compared to the first time point and the first time point has detectable autoantibody isotypes. This indicates worsening of the disease and/or lack of efficacious treatment. The increase is at least 5%, at least 10%, at least 20%, at least 30%, at least 50% at least 100%, at least 200%, at least 300%, at least 500%, at least 1000%, or more and including all the percentages between 5-1000%.

In one embodiment, there is a stable level of autoantibody isotypes, wherein the autoantibody isotypes detectable at the second and first time points are comparably similar within statistical analysis variances or deviation, about 1%, 2%, 3%, 4%, 5% and all the percentages between 1%-5% standard deviation from the level of auto-antibody isotype from the first time point. The stable level of autoantibody indicates stable disease, wherein the treatment has been of insufficient duration (i.e., that it should be continued if clinically indicated) or is ineffective.

In one embodiment of any one of the assays described, the assay further comprising diagnosing the likelihood of IBM based on a detectable presence or the level of the at least two antibody isotypes selected in the measuring step of the assay.

In one embodiment of any one of the assays described, a detectable presence of at least two autoantibody isotypes reactive against cN1A or protein fragment thereof, or cNIB, isoforms or protein fragment thereof indicates the likelihood of IBM in the subject.

In one embodiment of any one of the assays described, the detectable presence or the levels of the at least two autoantibody isotypes is based on the detection limit of the detection method utilized. In one embodiment of any of the assays described herein, the detectable presence of or the levels of reactive autoantibody isotypes selected are measured by Western blot analysis or ELISA. For example, the anti-cN1A or cN1B autoantibody isotypes can be analyzed in a 1D SDS PAGE gel followed by a Western blot analysis. The detectable presence of the autoantibody isotypes would be based on the detection limit of the Western blot on a 1D SDS PAGE gel.

In one embodiment of any one of the assays described, the assay has a sensitivity of at least 55%. In one embodiment of any one of the assays described, the assay has a sensitivity of ranging anywhere from 55%-80%. In other embodiments of any one of the assays described, the assay has a sensitivity of at least 57%, at least 59%, at least 60%, at least 62%, at least 64%, at least 65%, at least 67%, at least 69%, at least 70%, at least 72%, at least 74%, at least 75%, at least 77%, at least 79%, and at least 80%, including all the whole integer percent between 55% and 80%.

In one embodiment of any one of the assays described, the assay has a sensitivity of approximately 55%. In one embodiment of any one of the assays described, the assay has a sensitivity of ranging anywhere from approximately 55% to approximately 80%. In other embodiments of any one of the assays described, the assay has a sensitivity of approximately 57%, approximately 59%, approximately 60%, approximately 62%, approximately 64%, approximately 65%, approximately 67%, approximately 69%, approximately 70%, approximately 72%, approximately 74%, approximately 75%, approximately 77%, approximately 79%, and approximately 80%, including all the whole integer percent between approximately 55% and approximately 80%.

In one embodiment of any one of the assays described, the assay has a specificity of approximately 90%. In other embodiments of any one of the assays described, the assay has a specificity of approximately 91%, approximately 92%, approximately 93%, approximately 94%, approximately 95%, approximately 96%, approximately 97%, approximately 99%, approximately 100%, including all the whole integer percent between approximately 90% and approximately 100%.

In one embodiment of any one of the assays described, the assay has a specificity of no less than 90%. In other embodiments of any one of the assays described, the assay has a specificity of no less than 91%, no less than 92%, no less than 93%, no less than 94%, no less than 95%, no less than 96%, no less than 97%, no less than 99%, no less than 100%, including all the whole integer percent between 90% and 100%.

In one embodiment of any one of the assays described, the assay has a sensitivity of at least 55% and a specificity of no less than 90%. In one embodiment of any one of the assays described, the assay has a sensitivity of at least 70% and a specificity of no less than 90%.

In other embodiments of any one of the assays described, the assay has a sensitivity of at least 55% and a specificity of no less than 91%, a sensitivity of at least 55% and a specificity of no less than 93%, a sensitivity of at least 55% and a specificity of no less than 95%, a sensitivity of at least 60% and a specificity of no less than 90%, a sensitivity of at least 60% and a specificity of no less than 92%, a sensitivity of at least 60% and a specificity of no less than 94%, a sensitivity of at least 65% and a specificity of no less than 90%, a sensitivity of at least 65% and a specificity of no less than 91%, a sensitivity of at least 65% and a specificity of no less than 93%, a sensitivity of at least 70% and a specificity of no less than 91%, a sensitivity of at least 70% and a specificity of no less than 92%, a sensitivity of at least 70% and a specificity of no less than 93%, a sensitivity of at least 70% and a specificity of no less than 94%, and a sensitivity of at least 70% and a specificity of no less than 95%.

In one embodiment of any one of the assays described, the subject presents at least one symptom of a muscle disease. For example, muscular weakness, rigidity, loss of muscular control, myoclonus (twitching, spasming), and myalgia (muscle pain).

In one embodiment of any one of the assays described, the muscle disease is an inflammatory myopathy. For example, polymyositis (PM), dermatomyositis (DM), and inclusion body myositis (IBM).

In one embodiment of any one of the assays described, the at least one symptom is selected from the group consisting of progressive weakness in the muscles of the wrists and fingers, progressive weakness in the muscles that lift the front of the foot, progressive weakness in the muscles at the front of the thigh (quadriceps), and weakness of the swallowing muscles.

In one embodiment of any one of the assays described herein, the subject in need of diagnosing or prognosis evaluation presents symptoms of IBM, such as progressive muscle weakness, muscle pain and/or muscle fatigue.

In one embodiment of any one of the assays described herein, the PM, DM, other known autoimmune disorders has been ruled out in the subject, i.e., confirming that the patient is not suffering from PM, DM, or Myasthenia gravis etc.

In one embodiment of any one of the assays described, the subject in need of diagnosing has trouble with gripping, or has frequent stumbles, or has trouble swallowing due to weakness of the swallowing muscles.

In one embodiment of any one of the assays described, the subject in need of diagnosing or prognosis evaluation has not had an electromyogram and/or a muscle biopsy.

In one embodiment of any one of the assays described herein, the subject is suspected of having IBM.

In one embodiment of any one of the assays described herein, the IBM is idiopathic. In one embodiment, the IBM is idiopathic prior to the present disclosed assays. Idiopathic IBM arises spontaneously or is from an obscure or unknown cause. In one embodiment of any method or assay described herein, the patient has not had a muscle biopsy performed.

In one embodiment of any one of the assays described, the subject is a human. In one embodiment, the patient is a human over 40 years old. In another embodiment, the subject is a human over 50 years old. In one embodiment, the human subject is male. In one embodiment, the human subject is male and over 40 years old. In one embodiment, the human subject is female and over 40 years old.

In one embodiment of any one of the assays described, the autoantibodies are from a blood sample of the patient. Accordingly, in one embodiment of any one of the assay described, the biological sample is a blood sample. In another embodiment of any one of the assay described, the blood sample is plasma or serum. Methods of preparing of plasma or serum from a blood sample are known in the art. A skilled artisan can obtain the plasma or serum by any method known in the art, e.g., by centrifugation of a blood sample in the presence of an anti-coagulant to obtain plasma.

In one embodiment of any one of the assays described, the assay comprises measuring more than two antibody isotypes selected in the measuring step of the assay. In one embodiment of any one of the assays described, the assay comprises measuring two antibody isotypes selected in the measuring step of the assay. In one embodiment of any one of the assays described, the assay comprises measuring at least three antibody isotypes selected in the measuring step of the assay. In one embodiment of any one of the assays described, the assay comprises measuring three antibody isotypes selected in the measuring step of the assay. In one embodiment of any one of the assays described, the assay comprises measuring at least four antibody isotypes selected in the measuring step of the assay. In one embodiment of any one of the assays described, the assay comprises measuring four antibody isotypes selected in the measuring step of the assay. In one embodiment of any one of the assays described, the assay comprises measuring at least five antibody isotypes selected in the measuring step of the assay. In one embodiment of any one of the assays described, the assay comprises measuring five antibody isotypes selected in the measuring step of the assay.

In another embodiment, the antibody isotypes are selected from the group selected from IgG1, IgG2a, IgG2b, IgG3, IgG4, IgM, IgA1, IgA2, IgD, and IgE.

In some embodiments of any one of the assays described, the at least two isotypes are the combinations as follows: IgG and IgM; IgG and IgA; IgG and IgD; IgG and IgE; IgA and IgM; IgA and IgE; IgA and IgD; IgD and IgM; IgD and IgE; and IgM and IgE.

In some embodiments of any one of the assays described, the at least two isotypes are the combinations as follows: IgG, IgA and IgM; IgG, IgA and IgE; IgG, IgA and IgD; IgG, IgM and IgE; IgG, IgM and IgD; IgG, IgE and IgD; IgG, IgE and IgM; IgM, IgE and IgD; IgA, IgE and IgD; IgM, IgA and IgE; IgM, IgA and IgD; and IgD, IgE and IgG;

In some embodiments of any one of the assays described, the at least two isotypes are the combinations as follows: IgG, IgE, IgA and IgM; IgG, IgD, IgA and IgM; IgG, IgE, IgD and IgM; IgG, IgE, IgA and IgD; and IgD, IgE, IgA and IgM.

In another embodiment of any one of the assays described, the at least two isotypes are the combinations as follows: IgE and IgD; IgG, IgA, and IgD; IgG, IgA, and IgE; IgG, IgM, and IgD; IgG, IgM, and IgE; IgG, IgD, and IgE; IgG, IgA, IgM, and IgD; IgG, IgA, IgM, and IgE; IgG, IgA, IgD, and IgE; and IgG, IgM, IgD, and IgE.

In one embodiment of any one of the assays described, the at least two isotypes comprise any of the possible combinations derived from IgA, IgG, IgM, IgD, and IgE. In another embodiment of any one of the assays described, the at least two isotypes comprises any of the possible combinations derived from IgG1, IgG2a, IgG2b, IgG3, IgG4, IgM, IgA1, IgA2, IgD, and IgE.

In one embodiment of any one of the assays described, the at least two isotypes selected in the assay are IgA and IgM.

In one embodiment of any one of the assays described, the at least two isotypes selected in the assay are IgG and IgM.

In one embodiment of any one of the assays described, the at least two isotypes selected in the assay are IgG, IgA and IgM.

In one embodiment of any one of the assays described, the at least two isotypes selected in the assay does not include IgG. In one embodiment of any one of the assays described, the at least two isotypes selected in the assay does not include IgG1 and/or IgG2.

In one embodiment of any one of the assays described, IgM is one of the at least two isotypes selected.

In one embodiment of any one of the assays described, IgM is one of the at least two isotypes selected and IgG is not selected. In one embodiment of any one of the assays described, IgM is one of the at least two isotypes selected and IgG1 and IgG2 are not selected.

In one embodiment of any one of the assays described, the detectable presences of the at least two antibody isotypes selected indicate the likelihood of IBM in the subject.

In one embodiment of any one of the assays described, the assay further comprises comparing the level of the at least two antibody isotypes selected to respective antibody isotypes reference levels to determine the likelihood of IBM in the subject.

In one embodiment of any one of the assays described, the level of the at least two isotypes that are at least 5% over that of respective antibody isotype reference levels indicate the likelihood of IBM.

In one embodiment of any one of the assays described, the respective antibody isotype reference levels are the levels of the selected antibody isotypes that are reactive against the same cN1A, cN1B or protein fragment thereof in a biological sample of a healthy subject not having IBM, wherein the biological samples are the same for both the healthy subject and subject in need of diagnosis.

In one embodiment of any one of the assays described, the respective antibody isotype reference levels are the average levels of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis.

In one embodiment of any one of the assays described, the respective antibody isotype reference levels are the average levels plus one or two or three standard deviations of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis.

In one embodiment of any one of the assays described, the respective antibody isotype reference levels are the normalized levels of the selected antibody isotypes that are reactive against the same cN1A, cN1B or fragment thereof in a biological sample of a healthy subject not having IBM, wherein the normalization is performed against a level of albumin in the biological sample of a healthy subject not having IBM, wherein the biological samples are the same for both the healthy subject and subject in need of diagnosis.

In one embodiment of any one of the assays described, the healthy subject does not have IBM. In another embodiment of any one of the assays described, the healthy subject does not an inflammatory myopathy and/or a muscle disease. In another embodiment of any one of the assays described, the healthy subject does not have IBM but have a myopathy, and/or a muscle disease.

In one embodiment of any one of the assays described, the healthy subject does not having IBM, and/or an inflammatory myopathy, and/or a muscle disease.

In one embodiment of any one of the assays described, the detectable presences are determined by the detection limit of the measuring method used in measuring step b.

In one embodiment of any one of the assays described herein, the reference level is the level of an autoantibody isotype that are reactive and bind to a CN1A or a CN1B, or the peptide fragment thereof, or an isolated peptide, or a fusion protein described herein, the autoantibody isotype being from a blood sample of a healthy subject free of IBM or any inflammation myopathy or a muscle disease. In another embodiment, the reference level is obtained from subjects who do not have IBM but have other forms of myopathy or a muscle disease. In another embodiment, the reference level is obtained from a mixture of healthy subjects who do not have IBM or any other or any inflammation myopathy or a muscle disease and subjects who do not have IBM but have other forms of myopathy or a muscle disease. In one embodiment, the reference level is the threshold level described herein.

In one embodiment of any one of the assays described herein, the amount of anti-CN1A or anti-CN1B autoantibody isotypes in a healthy non-IBM individual or a population of healthy non-IBM individuals as determined by conventional ELISA or Western blot can be considered as the background or reference level. The collected blood sample, e.g., sera or plasma, from the healthy non-IBM individuals are diluted 1:100 and used in Western blot analysis. The intensity of visible bands on the blots is quantified by densitometry. From this densitometry data, the average value and the one order of standard deviation are computed using known statistical analysis method.

In one embodiment of any one of the assays described herein, the reference level is the average level obtained for a population of healthy subject free of any inflammation conditions. The following is an example for obtaining a reference level from a population of 25 healthy human subjects and should not be construed as limited to this example and methodology. Blood samples are taken from 25 healthy human subjects. The blood samples are measured for the level of autoantibody isotype binding to CN1A or the CN1B, or the peptide fragment thereof, or an isolated peptide, or a fusion protein described herein. The levels of autoantibodies can be measured by a number of immunological assay methods known in the art, e.g., quantitative Western blots and ELISA. The levels obtained for these 25 subjects are then added together and averaged, giving an average reference level. The size of the population from which the average reference level is obtained can vary anywhere from 3 to 100 or more, e.g., 500 subjects. The statistics, the average value and one order of standard deviation can be uploaded to the computer system and data storage media described herein.

In one embodiment of any one of the assays described herein, albumin levels in the biological samples are also measured and used to normalized the measured levels of autoantibody isotypes selected.

In one embodiment of any one of the assays described herein, the reference level is the detection limit of the measuring method used to measure the presence/absence or the level of antibody isotype immunoreactive binding to the CN1A or the CN1B, or the peptide fragment thereof, or an isolated peptide, or a fusion protein described herein. Accordingly, in one embodiment, a level of autoantibody isotypes above the detection limit of an immunological assay method used indicates the likelihood that the patient has IBM. For an example, the reference level is the detection limit of a Western blot analysis. A level of autoantibody isotypes above this detection limit of a Western blot analysis indicates the likelihood that the patient has IBM. It is assumed that the reagents and conditions of the immunological assay method used for the patient and the reference levels are kept the same. Similarly for the methods for evaluating treatment efficacy, the assays for measuring the levels of autoantibodies at the various time points are the same. As another example, if the immunological assay is an ELISA, then the reference level is the detection limit of an ELISA conducted or performed under similar assay conditions. A level of autoantibody isotypes above this detection limit of an ELISA indicates the likelihood that the patient has IBM.

In one embodiment of any one of the assays described, the measuring method comprises the steps of (a) contacting the biological sample (e.g. blood sample) from the subject with a cN1A, cN1B or protein fragment thereof or fusion proteins; (b) forming an antibody-protein complex between the antibody isotype present in the biological sample with the cN1A, cN1B or protein fragment thereof; (c) washing to remove any unbound antibody isotypes; (d) adding at least two detection antibodies that are labeled and are respectively reactive to the at least two antibody isotypes selected for the assay from the biological sample of the subject in order to detect the antibody-protein complex formed in step b; (e) washing to remove any unbound labeled detection antibodies; and (f) converting the label of the at least two detection antibodies to detectable signals, wherein a detectable signal for each of the at least two detection antibodies indicates the presence or level of respective anti-cN1A or anti-cN1B autoantibody isotype in the biological sample of the subject.

In one embodiment of any one of the assays described herein, the number of distinct and different detection antibodies matches the number of antibody isotype selected in the assay.

In one embodiment of any one of the assays described herein, the detection antibody is labeled by covalently linking to an enzyme, labeled with a fluorescent compound or metal, or labeled with a chemiluminescent compound. The labeling can be performed by any method known in the art.

In one embodiment of any one of the assays described herein, the cN1B is selected from any one of the known isoforms of NT5CN1B described herein.

In one embodiment of any one of the assays described herein, the protein fragment of cN1A or cN1B comprises at least 6 contiguous amino acid residues and is not a full-length cN1A, a full-length cN1B polypeptide or isoforms thereof.

In one embodiment of any one of the assays described herein, the protein fragment of NT5C1A or NT5CN1B is an isolated peptide selected from the group consisting of

(SEQ. ID. NO: 1)
AKIFYDNLAPKKKPKSPKPQNAVTIAVSSRALFRMD,
(SEQ. ID. NO: 2)
RALFRMDEEQQIYTEQGVEEYVRYQLEHENEPFSPG,
(SEQ. ID. NO: 3)
SQLRVAFDGDAVLFSDESERIVKAHGLDRFFEHEKA,
(SEQ. ID. NO: 4)
KIRPHIFFDDQMFHVAGAQEMGTVAAHVPYGVAQTP,
(SEQ. ID. NO: 5)
HVAGAQEMGTVAAHVPYGVAQTPRRTAPAKQAPSAQ,
(SEQ. ID. NO: 6)
SQWSRISRSPSTKAPSIDEPRSRNTSAKLPSSSTSS,
(SEQ. ID. NO: 7)
DGDAVLFSDESEHFTKEHGLDKFFQYDTLCESKPLA,
(SEQ. ID. NO: 8)
DGDAVLFSDESERIVKAHGLDRFF,
(SEQ. ID. NO: 9)
DGDAVLFSDESEHFTKEHGLDKFF,
(SEQ. ID. NO: 10)
GDAVLFSDESE,
and
(SEQ. ID. NO: 11)
HGLD(R/K)FF.

In one embodiment of any of the assays described herein, the protein fragment of cN1A or cN1B is fused or conjugated to a heterologous protein (i.e., a non-cN1A or cN1B protein) to form a fusion or chimeric protein such that the fusion protein is not a full-length cN1A, a full-length cN1B polypeptide or isoforms thereof. In some embodiments, the protein fragment of cN1A or cN1B is fused to a polymer. Methods of making such protein fragment of cN1A or cN1B or isoforms thereof, or making of isolated peptides of cN1A or cN1B or isoforms thereof, or making such fused or conjugated to a heterologous protein of cN1A or cN1B or isoforms thereof, are fully described in the PCT Application No: PCT/US2012/37275 which is hereby incorporated by reference in its entirety.

In one embodiment of any one of the assays described, the measurement method is an immunoassay. In one embodiment, the immunoassay is a serological immunoassay. In one embodiment of any one of the assays described, the immunoassay is an enzyme-linked immunosorbent assay. In one embodiment, the immunoassay comprises a formation of at least two distinct antibody-protein or antibody-peptide complexes.

In one embodiment of any one of the assays described, the immunoassay is an automated immunoassay.

In one embodiment of any one of the assays described, the measuring is performed with the use of a mass spectrometer.

In one embodiment of any one of the assays described, the measuring comprises detecting a detectable signal. A detectable signal indicates likelihood of IBM in the subject.

In one embodiment of any one of the assays described, the assay is performed by way of a point-of-care testing (POCT). In one embodiment of any one of the assays described, the measurement method is by way of is a lateral flow tests or immunoassay (LFIA) test strip.

In one embodiment of any one of the assays described, the assay is performed by a dipstick, a test strip, a microfluidic chip, a multi-well plate or a cartridge.

In one embodiment of any one of the assays described, the cN1A, cN1B or the isoform thereof, or the peptide fragment thereof, or the isolated peptide, or the fusion protein used in the assay is deposited or immobilized on a solid support.

In one embodiment of any one of the assays described, the isolated peptide is placed, deposited, immobilized or conjugated to a solid support. The support is in the format of a dipstick, a test strip, a latex bead, a microsphere, a multi-well plate or a cartridge.

In one embodiment of any one of the assays described, the assay comprises at least one solid support. Any solid support can be used, including but not limited to, nitrocellulose membrane, nylon membrane, solid organic polymers, such as polystyrene, or laminated dipsticks such as described in U.S. Pat. No. 5,550,375. The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. The “dip stick” technology can be easily adapted for the embodiments described herein by one skilled in the art. In embodiments of the assays described herein, the antigen for the autoantibody isotypes is a CN1A or CN1B, or protein fragment thereof, or an isolated peptide, or a fusion protein described herein. The antigen is deposited on the support and the various anti-CN1A autoantibody isotypes are detected by any method known in the art, e.g., by a detection antibody specific to each antibody isotype. In one embodiment, the assays described herein further comprise use of at least two different detection antibodies for the at least two antibody isotypes selected in the assay. In one embodiment, the detection antibody is detectably labeled.

In one embodiment, the isolated peptide derived from CN1A or CN1B is covalently linking to a label, e.g., with a fluorescent compound or metal, with latex that can be colored, with biotin, with proteins, with enzymes, or with a chemiluminescent compound. The isolated peptide is capable of complexing with the autoantibody isotypes from IBM patients. The following are only exemplary and should not be construed as limited to these. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises an alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid.

The immunoreactivity with autoantibody isotypes in the biological sample, e.g., the plasma, of a patient having IBM can be tested by any method known in the art, e.g., by dot blots, peptide gel Western analysis or by ELISA.

In one embodiment of any one of the assay described, the assay further comprises selecting a subject suspected of having IBM. In one embodiment, the subject suspected of having IBM presents at least one symptom known to be associated with IBM. In another embodiment, the subject presents skeletal muscle pain and/or muscle fatigue. In another embodiment, the subject presents skeletal muscle weakness. A skilled physician can evaluate with any basic muscle strength test known in the art.

In another embodiment of any one of the assays described, the assay further comprises selecting a subject in need of diagnosing whether the subject has IBM.

In another embodiment of any one of the assays described, the assay further comprises selecting a subject who is in need of diagnosing whether the subject has IBM and has tested negative for anti-cN1A or anti-cN1B autoantibody isotype IgG.

In one embodiment of any one of the assays described, the assay further comprises selecting the subject for treatment of IBM without having to perform an electromyogram and/or a muscle biopsy on the subject when there is detectable presence or the level of the at least two antibody isotypes selected in measuring step.

In one embodiment of any one of the assays described, the assay further comprises selecting the subject for treatment of IBM without having to perform an electromyogram and/or a muscle biopsy on the subject when the levels of the at least two isotypes selected in the measuring step are at least 5% over that of respective antibody isotype reference levels.

In one embodiment of any method described herein, the detecting or measuring of autoantibody isotypes comprises one dimension SDS-PAGE and Western blot analysis. In another embodiment of any method described herein, the detecting or measuring of autoantibody isotypes comprises an enzyme-linked immunoadsorbent assay (ELISA).

Also provided herein are methods of treatment of inclusion body myositis (IBM) in a subject in need thereof, the methods comprising administering an immunosuppressive therapy to a subject having a detectable presence or a level of at least two antibody isotypes that are reactive against a cytosolic 5′-nucleotidase 1A protein (cN1A) or a protein fragment thereof, or a cytosolic 5′-nucleotidase 1B (cN1B) protein or isoforms thereof or a protein fragment thereof, wherein the antibodies isotypes are selected from the group selected from IgG, IgM, IgA, IgD, and IgE.

In some embodiments of these methods and all such methods described herein, the methods further comprise comparing the levels of the at least two antibody isotypes in a second time point, wherein a decrease in the level of the antibody isotypes taken at the second time point indicates that the therapy is effective, wherein an increase in the level of the antibodies taken at the second time point are approximately the same indicates that the treatment is not effective, and wherein when the level of antibodies in the second time point decreases to below a detection limit indicates that the subject is in remission.

### CN1A, CN1B, Isoforms of CN1B and Peptides

The human cytosolic 5′-nucleotidase 1A (EC=3.1.3.5) is an enzyme that dephosphorylates the 5′ and 2′(3′)-phosphates of deoxyribonucleotides and has a broad substrate specificity. The enzyme helps to regulate adenosine levels in heart during ischemia and hypoxia. It is highly expressed in skeletal muscle and has been detected at intermediate levels in heart, brain, kidney and pancreas. The full-length CN1A polypeptide has 368 amino acid residues. Alternate names of CN1A include cN-1A, cN1A, NT5C1A and cN-1. The UNIPROTEIN identifier for CN1A is Q9BXI3. The GENBANK™ Accession number CN1A is NP_{—} 115915.1. (SEQ. ID. NO:12).

MEPGQPREPQEPREPGPGAETAAAPVWEEAKIFYDNLAPKKKPKSPKPQ
NAVTIAVSSRALFRMDEEQQIYTEQGVEEYVRYQLEHENEPFSPGPAFP
FVKALEAVNRRLRELYPDSEDVFDIVLMTNNHAQVGVRLINSINHYDLF
IERFCMTGGNSPICYLKAYHTNLYLSADAEKVREAIDEGIAAATIFSPS
RDVVVSQSQLRVAFDGDAVLFSDESERIVKAHGLDRFFEHEKAHENKPL
AQGPLKGFLEALGRLQKKEYSKGLRLECPIRTYLVTARSAASSGARALK
TLRSWGLETDEALFLAGAPKGPLLEKIRPHIFFDDQMFHVAGAQEMGTV
AAHVPYGVAQTPRRTAPAKQAPSAQ

The human cytosolic 5′-nucleotidase 1B (EC=3.1.3.5) is an enzyme related to CN1A. There are at least four isoforms of CN1B and they are the result of alternate splicing of the primary transcript. The isoforms have about 65% identity to CN1A. Like CN1A, CN1B dephosphorylates the 5′ and 2′(3′)-phosphates of deoxyribonucleotides and helps to regulate adenosine levels. It is highly expressed in testis, placenta and pancreas, and is also detected at lower levels in heart, kidney, liver and lung. Alternate names of CN1B include autoimmune infertility-related protein, cN1B, NT5C1B, cN-1B, AIRP and FKSG85.

Alternative splicing of the human CN1B gene produces several isoforms, 1-5. UNIPROTEIN identifiers for the isoforms 1-4 are Q96P26-1 (SEQ. ID. NO:13), Q96P26-2 (SEQ. ID. NO:14), Q96P26-2 (SEQ. ID. NO:15), and Q96P26-4 (SEQ. ID. NO:16) respectively. GENBANK Accession numbers of the isoforms are NP_001002006, NP_001186015.1, NP_001186016.1, NP_001186017.1, NP_001186033.1, NP_001002006.1, NM_001002006.2 and NP_150278.2.

Q96P26-1
(SEQ. ID. NO: 13)
MSQTSLKQKKNEPGMRSSKESLEAEKRKESDKTGVRLSNQMRRAVNPNH
SLRCCPFQGHSSCRRCLCAAEGTALGPCHTIRIYIHMCLLWEQGQQITM
MRGSQESSLRKTDSRGYLVRSQWSRISRSPSTKAPSIDEPRSRNTSAKL
PSSSTSSRTPSTSPSLHDSSPPPLSGQPSLQPPASPQLPRSLDSRPPTP
PEPDPGSRRSTKMQENPEAWAQGIVREIRQTRDSQPLEYSRTSPTEWKS
SSQRRGIYPASTQLDRNSLSEQQQQQREDEDDYEAAYWASMRSFYEKNP
SCSRPWPPKPKNAITIALSSCALFNMVDGRKIYEQEGLEKYMEYQLTNE
NVILTPGPAFRFVKALQYVNARLRDLYPDEQDLFDIVLMTNNHAQVGVR
LINSVNHYGLLIDRFCLTGGKDPIGYLKAYLTNLYIAADSEKVQEAIQE
GIASATMFDGAKDMAYCDTQLRVAFDGDAVLFSDESEHFTKEHGLDKFF
QYDTLCESKPLAQGPLKGFLEDLGRLQKKFYAKNERLLCPIRTYLVTAR
SAASSGARVLKTLRRWGLEIDEALFLAGAPKSPILVKIRPHIFFDDHMF
HIEGAQRLGSIAAYGFMSQTSLKQKKNEPGMRSSKESLEAEKRKESDKT
GVRLSNQGSQESSLRKTDSRGYLVRSQWSRIS
Q96P26-2
(SEQ. ID. NO: 14)
RSPSTKAPSIDEPRSRNTSAKLPSSSTSSRTPSTSPSLHDSSPPPLSGQ
PSLQPPASPQLPRSLDSRPPTPPEPDPGSRRSTKMQENPEAWAQGIVRE
IRQTRDSQPLEYSRTSPTEWKSSSQRRGIYPASTQLDRNSLSEQQQQQR
EDEDDYEAAYWASMRSFYEKNPSCSRPWPPKPKNAITIALSSCALFNMV
DGRKIYEQEGLEKYMEYQLTNENVILTPGPAFRFVKALQYVNARLRDLY
PDEQDLFDIVLMTNNHAQVGVRLINSVNHYGLLIDRFCLTGGKDPIGYL
KAYLTNLYIAADSEKVQEAIQEGIASATMFDGAKDMAYCDTQLRVAFDG
DAVLFSDESEHFTKEHGLDKFFQYDTLCESKPLAQGPLKGFLEDLGRLQ
KKFYAKNERLLCPIRTYLVTARSAASSGARVLKTLRRWGLEIDEALFLA
GAPKSPILVKIRPHIFFDDHMFHIEGAQRLGSIAAYGFNKKFSS
Q96P26-3
(SEQ. ID. NO: 15)
MQENPEAWAQGIVREIRQTRDSQPLEYSRTSPTEWKSSSQRRGIYPAST
QLDRNSLSEQQQQQREDEDDYEAAYWASMRSFYEKNPSCSRPWPPKPKN
AITIALSSCALFNMVDGRKIYEQEGLEKYMEYQLTNENVILTPGPAFRF
VKALQYVNARLRDLYPDEQDLFDIVLMTNNHAQVGVRLINSVNHYGLLI
DRFCLTGGKDPIGYLKAYLTNLYIAADSEKVQEAIQEGIASATMFDGAK
DMAYCDTQLRVAFDGDAVLFSDESEHFTKEHGLDKFFQYDTLCESKPLA
QGPLKGFLEDLGRLQKKFYAKNERLLCPIRTYLVTARSAASSGARVLKT
LRRWGLEIDEALFLAGAPKSPILVKIRPHIFFDDHMFHIEGAQRLGSIA
AYGFNKKFSS
Q96P26-4
(SEQ. ID. NO: 16)
MSQTSLKQKKNEPGMRSSKESLEAEKRKESDKTGVRLSNQMRRAVNPNH
SLRCCPFQGHSSCRRCLCAAEGTALGPCHTIRIYIHMCLLWEQGQQITM
MRGSQESSLRKTDSRGYLVRSQWSRISRSPSTKAPSIDEPRSRNTSAKL
PSSSTSSRTPSTSPSLHDSSPPPLSGQPSLQPPASPQLPRSLDSRPPTP
PEPDPGSRRSTKMQENPEAWAQGIVREIRQTRDSQPLEYSRTSPTEWKS
SSQRRGIYPASTQLDRNSLSEQQQQQREDEDDYEAAYWASMRSFYEKNP
SCSRPWPPKPKNAITIALSSCALFNMVDGRKIYEQEGLEKYMEYQLTNE
NVILTPGPAFRFVKALQYVNARLRDLYPDEQDLFDIVLMTNNHAQVGVR
LINSVNHYGLLIDRFCLTGGKDPIGYLKAYLTNLYIAADSEKVQEAIQE
GIASATMFDGAKDMAYCDTQLRVAFDGDAVLFSDESEHFTKEHGLDKFF
QYDTLCESKPLAQGPLKGFLEDLGRLQKKFYAKNERLLCPIRTYLVTAR
SAASSGARVLKTLRRWGLEIDEALFLAGAPKSPILVKIRPHIFFDDHMF
HIEGAQRKSLGWMS

In one embodiment, the CN1A and CN1B are mammalian proteins. In one embodiment, the CN1A and CN1B are human CN1A and human CN1B respectively.

In one embodiment of any one of the assays described, an isolated peptide derived from the sequence of a cytosolic 5′-nucleotidase 1A protein (CN1A), a cytosolic 5′-nucleotidase 1B protein (CN1B) or an isoform thereof is bound by the autoantibody isotypes in a biological sample, e.g., blood, of a patient having inclusion body myositis (IBM). One characteristic of the peptide is its binding to autoantibodies from a blood sample e.g., plasma or serum, from a patient having IBM. In one embodiment, the patient is one who has had a muscle biopsy performed to definitively confirm the presence of IBM. In another embodiment, the patient is one who has all the symptoms known associated with IBM and also have all other known possible causes systematically ruled out. In another embodiment, the patient is one who has more than one of the symptoms known associated with IBM and also have all other known possible causes systematically ruled out.

In one embodiment, the CN1A, CN1B or isoforms thereof is derived from a mammal. In one embodiment, the CN1A, CN1B or isoforms thereof is derived from a human.

Such isolated peptides are useful for detecting autoantibodies present in a blood sample of a patient suspected of having IBM. Such isolated peptides are useful for the development of non-invasive diagnosis or detection assays, kits and systems for IBM.

### Inclusion Body Myositis (IBM)

The inflammatory myopathies are autoimmune diseases of skeletal muscle, and consist of three major subtypes: dermatomyositis (DM), polymyositis (PM), and inclusion body myositis (IBM). Circulating autoantibodies have been detected in DM and PM and sought in IBM. Since 1984, IBM has been believed to be a cytotoxic T-cell mediated disease with no humoral autoimmunity (4). Microarray studies reported in 2001, surprisingly showed that the most abundantly present transcripts in IBM muscle samples compared to normal muscle were immunoglobulin transcripts, unique to the B cell lineage. In 2010, it has been demonstrated that there are IgG anti-cN1A autoantibodies in IBM patients but not PM and DM patients.

Aspects of the technology described herein relate to the diagnosis, prognosis, and treatment efficacy evaluation of IBM and also for treatment of IBM. As used herein, “inclusion body myositis” or “IBM” refers to an inflammatory myopathy in which the muscle cells of the subject comprise inclusion bodies. Methods of differential diagnosis of IBM include evaluating the following: age (typically >30 years); duration of illness (typically >6 months, mostly over months or years); gender (typically males>females 2:1); signs and symptoms of progressive muscle weakness; and an assortment of laboratory tests. In general, the clinical evaluation would include, but is not limited to, physical examination, measurement of electrical activity in the muscles of the subject (e.g. a nerve conduction test or an electromyogram (EMG)), a muscle biopsy (e.g. to look for signs of inflammation, muscle fiber death, vascular deformities and/or inclusion bodies), ultrasound to look for muscle inflammation, magnetic resonance imaging to reveal abnormal muscle and evaluate muscle disease, antibody tests, and a family history. IBM primarily affects men over the age of 50 but can affect younger subjects and women. Symptoms of IBM that can be useful in making a diagnosis of IBM include, but are not limited to, muscle weakness, muscle atrophy, or pain; falling and tripping, difficulty swallowing (dysphagia) occurs in approximately half of IBM cases.

In some embodiments, muscle weakness can begin in the wrists, fingers, and quadriceps; weakness of the swallowing muscles can occur. In some embodiments, IBM can be distinguished from other inflammatory myopathies (e.g. PM and DM) by the subject having normal or only mildly elevated creatine kinase levels in the bloodstream, whereas subjects with PM or DM typically have extremely elevated levels of creatine kinase. In some embodiments, IBM can also be distinguished from PM and DM by determining that the heart and lung muscles are not affected.

A number of serological testing are performed to exclude other conditions that affects muscles. Detailed testing of the antinuclear antibodies (ANA), when present, is important in identifying other overlap syndromes, most often those with another autoimmune disorder. About 30% of patients have myositis-specific autoantibodies: antibodies to aminoacyl-tRNA synthetases (anti-synthetase antibodies), including anti-Jo-1; antibodies to signal recognition particle (SRP—anti-SRP antibodies); and antibodies to Mi-2, a nuclear helicase. The relationship between these autoantibodies and disease pathogenesis remains unclear, although antibody to Jo-1 is a significant marker for fibrosing alveolitis, pulmonary fibrosis, arthritis, and Raynaud's syndrome.

Factors which can put a subject at risk for developing IBM include, but are not limited to, infection with a virus, e.g. the HIV virus, the HTLV-1 virus, or the Coxsackie B virus; exposure to certain drugs (e.g. carticaine, penicillamine, interferon-alpha, cimetidine, carbimazole, phenytoin, growth hormone, and the vaccine for hepatitis B), and a family history of IBM.

Current treatments for IBM are primarily aimed at symptomatic relief and support of musculature. These treatments are limited in effectiveness and they include, without being limited to, immunosupressives, corticosteroids, and supportive treatment for symptoms. Supportive treatment for symptoms includes, but is not limited to physical therapy, exercise, heat therapy (e.g. microwave and ultrasound), orthotics and assistive devices, and rest.

### Diagnostic Kits

In one embodiment, provided herein are kits that comprise an antigen and at least two detection antibodies that are specific for two different antibody isotypes, wherein the antibodies isotypes are selected from the group selected from IgG, IgM, IgA, IgD, and IgE, and wherein the antigen is selected from a CN1A or CN1B, or protein fragment thereof, or an isolated peptide, or a fusion protein described herein. In other words, the kit provide reagents for detecting the presence of antibody IgG isotype, antibody IgM isotype, antibody IgA isotype, antibody IgD isotype, and antibody IgE isotype from a biological sample. In some embodiments, the kit comprises at least three detection antibodies that are specific for three different antibody isotypes, or the kit comprises at least four detection antibodies that are specific for four different antibody isotypes, or the kit comprises at least five detection antibodies that are specific for five different antibody isotypes, wherein the antibodies isotypes are selected from the group selected from IgG, IgM, IgA, IgD, and IgE.

In one embodiment of the kit, the detection antibody that is specific for an antibody isotype is labeled. In one embodiment, the detection antibody produces a detectable signal. For example, if the autoantibodies are from a subject that is a human, the detection antibody is anti-human IgG or anti-human IgM or anti-human IgA antibody. In one embodiment, the kit can include a second labeled CN1A or CN1B, or protein fragment thereof, or an isolated peptide, or a fusion protein described herein, the label produces a detectable signal

Diagnostic kits for carrying out the assays described herein are produced in a number of ways. In one embodiment, the diagnostic kit comprises (a) at least one of a CN1A or CN1B, or protein fragment thereof, or an isolated peptide, or a fusion protein described herein, the protein or peptide or fusion protein may or may not be conjugated to a solid support and (b) at least two different detection antibodies, each being conjugated to a detectable group, and the detection groups are dissimilar. In one embodiment, the diagnostic kit further comprises other reagent(s). The reagents include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. Alternatively, a test kit contains (a) a control antibody that reacts against the CN1A or CN1B, or protein fragment thereof, or the isolated peptide, or the fusion protein supplied with the kit, and (b) an additional detection antibody conjugated to a detectable group.

The test kit may be packaged in any suitable manner, typically with all elements in a single container, optionally with a sheet of printed instructions for carrying out the test.

In one embodiment of the kit described herein, the kit comprises LFIA test strips.

In one embodiment of the kit described herein, the kit further comprises at least an agent or substrate for producing a detectable signal from each of the at least two different detection antibodies of the kit.

Examples of kits include but are not limited to ELISA assay kits, and kits comprising test strips and dipsticks. In an ELISA kit, an excess amount of CN1A or CN1B, or protein fragment thereof is immobilized on a solid support. A sample containing an unknown amount of autoantibodies to CN1A or CN1B, or protein fragment thereof, or an isolated peptide, or a fusion protein described herein is added to the immobilized antigen, resulting in the formation of a complex consisting of the protein and the antibody. The complex is detected by a labeled second antibody that is specific for an antibody from the patient, e.g., a detection antibody is specific for human antibodies and the patient is a human. The amount of label detected is a measure of the amount of autoantibody present in the blood sample.

In some embodiments of the kits described herein, the kit comprises a test strip (e.g., a LFIA test strip) or a dipstick.

In some embodiments of the kits described herein, the labeled detection antibodies are detectably labeled by enzyme labeling, fluorescent labeling, biotin labeling or radioisotope labeling. Other labels include but are not limited to colloidal gold and latex beads. The latex beads can also be colored. Methods of labeling antibodies are known in the art, for example, as described in “Colloidal Gold. Principles. Methods and Applications”, Hayat M A (ed.) (1989-91). Vols 1-3, Academic press, London; in “Techniques in Immunocytochemistry”, Bullock G R and Petrusz P (eds) (1982-90) Vols 1, 2, 3, and 4, Academic Press, London; in “Principles of Biological Microtechnique”, Baker J R (1970), Methuen, London; Lillie R D (1965), Histopathologic Technique and practical Histochemistry, 3rd ed, McGraw Hill, New York; Berryman M A, et al (1992), J. Histochem Cytochem 40, 6, 845-857, all of which are hereby incorporated by reference in their entirety.

In a typical colloidal gold labeling technique, the unique red color of the accumulated gold label, when observed by lateral or transverse flow along a membrane on which an antigen is captured by an immobilized antibody, or by observation of the red color intensity in solution, provides an extremely sensitive method for detecting sub nanogram quantities of proteins in solution. A colloidal gold conjugate consists of a suspension of gold particles coated with a selected protein or macromolecule (such as an antibody or antibody-based moiety). The gold particles may be manufactured to any chosen size from 1-250 nm. This gold probe detection system, when incubated with a specific target, such as in a tissue section, will reveal the target through the visibility of the gold particles themselves. For detection by eye, gold particles will also reveal immobilized antigen on a solid phase such as a blotting membrane through the accumulated red color of the gold sol. Silver enhancement of this gold precipitate also gives further sensitivity of detection. Suppliers of colloidal gold reagents for labeling are available from SPI-MARK™. Polystyrene latex Bead size 200 nm colored latex bead coated with antibody SIGMA ALDRICH®, Molecular Probes, Bangs Laboratory Inc., and AGILENT® Technologies.

In other embodiments of the kits described herein, at least one of the labeled detection antibodies is an enzyme-labeled antibody. The various anti-CN1A or anti-CN1B antibody isotypes that are bound and captured by the immobilized CN1A or CN1B, or protein fragment thereof, or an isolated peptide, or a fusion protein described herein on the solid support (e.g. microtiter plate wells) are identified by adding a chromogenic substrate for the enzyme conjugated to the labeled detection antibody, e. g. anti-human IgG, and color production detected by an optical device such as an ELISA plate reader. One skilled in the art would know to use different labeled detect labels to distinguish the different antibody isotypes IgG, IgM, IgA, IgD, and IgE, e.g., different chromogenic substrate for IgM and IgA in order to distinguish between the isotypes studied.

Other detection systems can also be used, for example, a biotin-streptavidin system. Quantification is determined using a streptavidin-peroxidase conjugate and a chromagenic substrate. Such streptavidin peroxidase detection kits are commercially available, e.g., from DAKO; Carpinteria, Calif.

Detection antibodies and CN1A or CN1B or protein fragment thereof can alternatively be labeled. For example, labeling can be achieved with any of a number of fluorescent compounds such as fluorescein isothiocyanate, europium, lucifer yellow, rhodamine B isothiocyanate (Wood, P. In: Principles and Practice of Immunoassay, Stockton Press, New York, pages 365-392 (1991)) for use in immunoassays. In conjunction with the known techniques for separation of antibody-antigen complexes, these fluorophores can be used to quantify the level of autoantibodies. The same applies to chemiluminescent immunoassay in which case antibody or desired antigen can be labeled with isoluminol or acridinium esters (Krodel, E. et al., In: Bioluminescence and Chemiluminescence: Current Status, John Wiley and Sons Inc. New York, pp 107-110 (1991); Weeks, I. et al., Clin. Chem., 29:1480-1483 (1983)). Radioimmunoassay (Kashyap, M. L. et al., J. Clin. Invest., 60:171-180 (1977)) is another technique in which detection antibody can be used after labeling with a radioactive isotope such as ¹²⁵I. Some of these immunoassays can be easily automated by the use of appropriate instruments such as the IMX™ (Abbott, Irving, Tex.) for a fluorescent immunoassay and Ciba Corning ACS 180™ (Ciba Corning, Medfield, Mass.) for a chemiluminescent immunoassay.

In some embodiments, the kits described herein further comprise standards of known amounts of CN1A or CN1B, or protein fragment thereof, or an isolated peptide, or a fusion protein described herein. In some embodiments, these standards of known amounts are deposited or immobilized on a solid support to form a calibration or titration standard devices for comparison with the device used with test samples. In one embodiment, the kit comprises calibration or titration standard devices having known amounts of antigen of the autoantibody of interest. For example, calibration or titration standard devices are strips with 0 ng, 0.5 ng, 1 ng, 2.5 ng, 5 ng, 10 ng, 20 ng, and 50 ng of antigen deposited or immobilized on a solid support.

In some embodiments, the kits described herein further comprise reference values of the levels of anti-CN1A or anti-CN1B antibodies isotypes IgG, IgM, IgA, IgD, and IgE. The reference values are average levels of anti-CN1A or CN1B antibodies isotypes in samples from a population of non-IBM healthy humans. Reference values can be provided as numerical values, or as standards of known amounts or titer of anti-CN1A or anti-CN1B antibodies isotypes presented in pg/ml to g/ml.

In some embodiments, the kits described herein further comprise at least one sample collection container for sample collection. Collection devices and container include but are not limited to syringes, lancets, BD VACUTAINER® blood collection tubes.

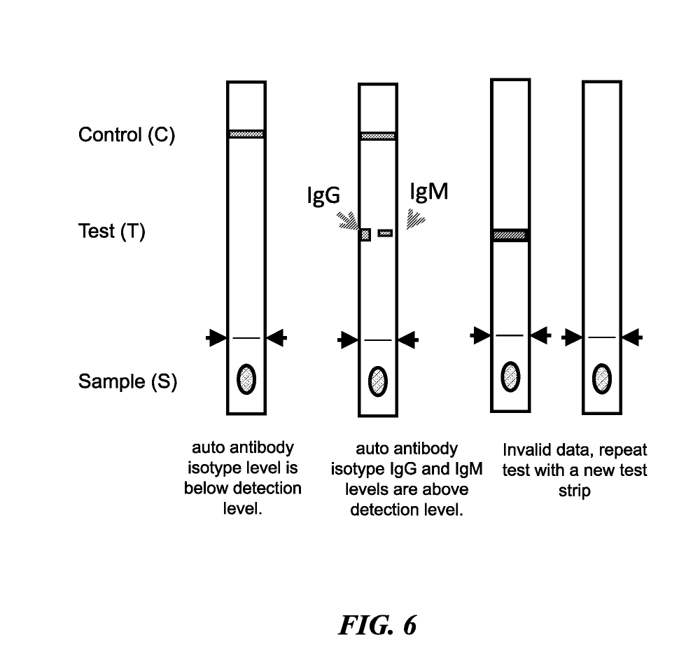

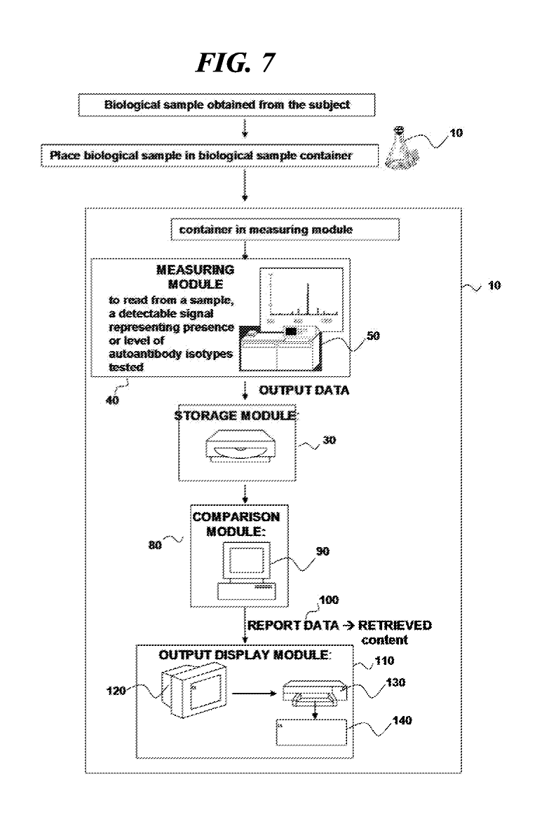

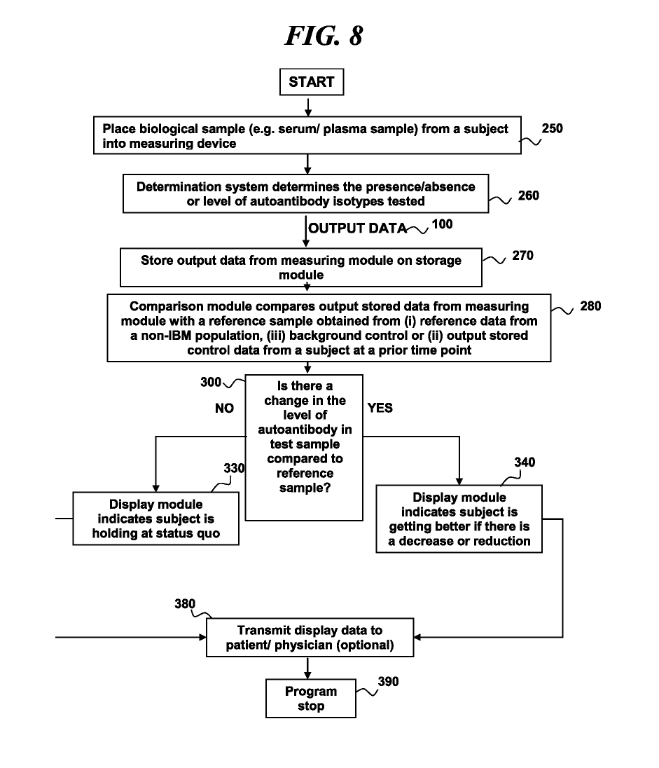

In some embodiments, the kits described herein further comprise instructions for using the kit and interpretation of results. For example, a chart showing FIG. 6 interpretation of results.

### Computer-Based Systems and Computer Data Storage

In one embodiment, provided herein is a system comprising a measuring module which measures autoantibody isotype information comprising at least two detectable signals from an immunoassay indicating the presence or level of autoantibodies isotypes from a biological sample, e.g., blood, obtained from a patient, autoantibodies isotypes are reactive to a CN1A or CN1B or a protein fragment thereof, or an isolated peptide, or a fusion protein described herein, wherein the antibodies isotypes are selected from the group selected from IgG, IgM, IgA, IgD, and IgE, and wherein each signal level correspond to an antibody isotype; a storage module configured to store data output from the measuring module; a comparison module adapted to compare the data stored on the storage module with at least two reference levels and to provide a retrieved content, wherein reference levels are the reference levels for the respective antibodies isotypes measured and are selected from the group selected from IgG, IgM, IgA, IgD, and IgE; and an output module for displaying the retrieved content for the user, wherein the retrieved content, the presence of detectable amount of at least two autoantibodies isotypes reactive against CN1A or CN1B or protein fragment, or an isolated peptide, or a fusion protein described herein, indicates that the patient has IBM or has a relapse of IBM. In other embodiments, the presence of detectable amount of at least three autoantibodies or at least four isotypes reactive against CN1A or CN1B or protein fragment, or an isolated peptide, or a fusion protein described herein, indicates that the patient has IBM or has a relapse of IBM.

In another embodiment, provided herein is a system for evaluating the efficacy of a treatment for a patient having IBM or to facilitate the prognosis evaluation of IBM in a patient, comprising: a measuring module configured to receive and output autoantibody isotype information from a biological sample, e.g., blood, obtained from a patient, wherein the autoantibodies information measures the level of auto antibodies isotypes that are reactive to a CN1A or CN1B or a protein fragment thereof, or an isolated peptide, or a fusion protein described herein, wherein the antibodies isotypes are selected from the group selected from IgG, IgM, IgA, IgD, and IgE; a storage module configured to store output information from the measuring module; a comparison module adapted to compare the data stored on the storage module with reference data, and to provide a comparison content, wherein the reference data comprises previous data from the same patient wherein the previous data had indicated detectable amounts of autoantibodies isotypes, and an output module for displaying the comparison content for the user, wherein if there is no detectable amount of autoantibodies isotypes reactive against CN1A or CN1B or a peptide fragment, or an isolated peptide, or a fusion protein described herein, then the patient is in remission or if there is a reduction of at least 5% compared to a prior reading or prior data, then the treatment is effective in the patient.

In one embodiment, provided herein is a computer readable storage medium comprising a storing data module containing data from a blood sample obtained from a subject that represents at least two signal levels from an immunoassay for autoantibodies isotypes that are reactive to a CN1A or CN1B or a protein fragment thereof, or an isolated peptide, or a fusion protein described herein, wherein the antibodies isotypes are selected from the group selected from IgG, IgM, IgA, IgD, and IgE, wherein each signal level correspond to an antibody isotype; a comparison module that compares the data stored on the storing data module with a reference data, wherein the reference data are the reference levels for the respective antibodies isotypes measured and are selected from the group selected from IgG, IgM, IgA, IgD, and IgE, and to provide a comparison content, and an output module displaying the comparison content for the user, wherein the presence of a detectable amount of at least two antibodies isotypes reactive against at least a CN1A or CN1B or a protein fragment thereof, or an isolated peptide, or a fusion protein of at least 5% relative to the reference data indicate that the subject has IBM or has a relapse of IBM.

In one embodiment of the system and storage medium described herein, the reference level comprises data from a population of non-IBM healthy individuals and/or non-IBM healthy individuals without any inflammatory conditions as described in this application. For example, the data are the detection signals obtained from the human sera or plasma from individuals of the population, from human sera or plasma at 1:100 dilution with 1× PBS and are immunoreactive with 0.5 g of a CN1A or CN1B or a protein fragment thereof, or an isolated peptide, or a fusion protein, wherein horse-radish peroxidase (HRP) labeled anti-human IgG antibody is the labeled detection antibody and the detection signal is chemiluminescence if IgG isotype is the selected antibody isotype. For the other antibody isotypes, IgM, IgA, IgD, and IgE, then horse-radish peroxidase labeled anti-human IgM antibody, HRP-labeled anti-human IgA antibody, HRP-labeled anti-human IgD antibody and HRP-labeled anti-human IgE antibody can be used respectively depending which antibody isotype is selected.

In some embodiments, the reference data comprises previous data from the same subject or patient wherein the previous data had indicated detectable amounts of autoantibodies.

In some embodiments, the reference data comprises previous data from the same subject or patient wherein the previous data had indicated no detectable amounts of autoantibodies.

Alternatively, the reference data comprises data from a population of non-IBM healthy individuals and/or non-IBM healthy individuals without any inflammatory conditions, the reference data for each of the antibody isotypes, IgG, IgM, IgA, IgD, and IgE is the average of the detection signals for the respective antibody isotype obtained by any known serological immunoassay using the human sera or plasma from non-IBM healthy individuals, and the detection signals correspond to the immunoreactivity of the sera or plasma with 0.5 g of a CN1A or CN1B or a protein fragment thereof.

Embodiments of the system and storage medium are described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer. In some embodiments, computer readable storage media include any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present technology. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the system and storage medium described herein include at minimum a measuring module #40, a storage module #30, a comparison module #80, and an output module #110. The functional modules can be executed on one, or multiple, computers, or by using one, or multiple, computer networks. The measuring module has computer executable instructions to provide e.g., expression information in computer readable form.

The measuring module #40 can comprise any system for detecting a signal representing expression level of auto-antibodies. Such systems can include any ELISA detection system and/or any Western blotting detection system.

The information determined in the measuring system can be read by the storage module #30. As used herein the “storage module” is intended to include any suitable computing or processing apparatus or other device configured or adapted for storing data or information. Examples of electronic apparatus suitable for use with the present technology include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon expression level or protein level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of the system and storage medium described herein, the reference data stored in the storage module to be read by the comparison module includes but are not limited to serological immunoassay, ELISA, and Western blot densitometry data obtained from a population of non-IBM subjects, a population of IBM subjects or expression data obtained from the same subject at a prior time point using the measuring module #40.

The “comparison module” #80 can use a variety of available software programs and formats for the comparison operative to compare expression data determined in the measuring module to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to normalized expression level of auto-antibodies, presence/absence of IBM in an individual, efficacy of treatment in an individual.

The comparison module, or any other module of the system and storage medium described herein, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executable will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GENBANK™ or Swiss Pro World Wide Web site).

The comparison module provides a computer readable comparison result that can be processed in computer readable form by predefined criteria, or criteria defined by a user, to provide a content-based in part on the comparison result that may be stored and output as requested by a user using an output module #110.

The content based on the comparison result, may be an expression value compared to a reference showing the presence/absence of IBM in an individual or the relapse or remission of IBM in a subject.

In one embodiment of any system described herein, the content based on the comparison result is displayed on a computer monitor #120. In one embodiment of any system described herein, the content based on the comparison result is displayed through printable media #130 and #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the system and storage medium described herein can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

Embodiments described herein therefore provide for systems (and computer readable media for causing computer systems) to perform methods for diagnosing IBM, assessing treatment efficacy of IBM, and/or monitoring recurrence of IBM in an individual.

Systems and computer readable media described herein are merely illustrative embodiments for detecting autoantibodies isotypes reactive against ˜0.5 g of a CN1A or CN1B, or a protein fragment thereof, or an isolated peptide, or a fusion protein described herein in an individual, and are not intended to be limiting in scope. Variations of the systems and computer readable media described herein are possible and are intended to fall within the scope of systems and computer readable media described herein.

The modules of the machine, or those used in the computer readable medium, may assume numerous configurations. For example, function may be provided on a single machine or distributed over multiple machines.

### Biological Sample Collection and Preparation

Collections of samples can be performed by methods well known to those skilled in the art.

For example, the patient's blood can be drawn by trained medical personnel directly into anti-coagulants such as citrate and EDTA. The whole blood can be separated into the plasma portion, the cells, and platelets portion by refrigerated centrifugation at 3500×G for 2 minutes. After centrifugation, the supernatant is the plasma.

Alternately, the serum can be collected from the whole blood. Collect the blood in a hard plastic or glass tube; blood will not clot in soft plastic. Draw 15 mL of whole blood for 6 mL of serum. The whole blood is allowed to stand at room temperature for 30 minutes to 2 hours until a clot has formed. Carefully separate clot from the sides of the container using a glass rod or wooden applicator stick and leave overnight at 4° C. After which, decant serum, centrifuge, and/or using a Pasteur pipette, remove serum into a clean tube. Clarify the serum by centrifugation at 2000-3000 rpm for 10 minutes. The serum is stored at −20° or −80° C. before analysis for auto-antibodies is performed. Detailed description of obtaining serum using collection tubes can be found in U.S. Pat. No. 3,837,376 and is hereby incorporated by reference in its entirety. Blood collection tubes can also be purchased from BD Diagnostic Systems, Greiner Bio-One, and Kendall Company.

### Detection of Autoantibodies

The detection of autoantibody isotypes against a CN1A or CN1B, or a protein fragment thereof, or an isolated peptide, or a fusion protein described herein in a biological sample, e.g., a blood, serum or plasma, of a patient can be detected by any method known in the art. In one embodiment, the levels of autoantibody isotypes in the biological samples of patients are detected by an immunoassay. Immunoassays include but are not limited to enzyme immunoassay (EIA), also called enzyme-linked immunosorbant assay (ELISA), radioimmunoassay (RIA), diffusion immunoassay (DIA), fluoroimmunoassay (FIA), chemiluminescent immunoassay (CLIA), counting immunoassay (CIA), lateral flow tests or immunoassay (LFIA), also known as lateral flow immunochromatographic assays, and magnetic immunoassay (MIA). In some embodiment, the immunoassay can be a quantitative or a semi-quantitative immunoassay or even a qualitative immunoassay configure to give a yes/no result.

An immunoassay is a biochemical test that measures the concentration of a substance in a biological sample, typically fluid sample such as serum, using the reaction of an antibody or antibodies to its antigen. The assay takes advantage of the specific binding of an antibody to its antigen. For the methods and assays described herein, specific binding of the autoantibody isotypes with respective proteins or protein fragments, or an isolated peptide, or a fusion protein described herein occurs in the immunoassay to form an autoantibody-protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries. For the methods and assays described herein, wherein the autoantibodies are detected by ELISA, a known amount of antigen (e.g., 0.5 g of a CN1A or CN1B or a protein fragment thereof, or an isolated peptide, or a fusion protein described herein) is affixed to a surface or solid support. Then the sample to be tested, e. g. blood, serum or plasma, suspected of containing autoantibodies, is washed over the surface so that the autoantibodies can bind to the immobilized antigen if the autoantibodies are present. The surface is washed to remove any unbound antibodies from the test sample and a detection antibody is applied to the surface. The detection antibody is specific to the antibodies from the subject and also specific to a particular antibody isotype. For example, if the subject is a human, the detection antibody could be an anti-human IgG antibody, anti-human IgM, antibody, anti-human IgA antibody, anti-human IgD antibody or anti-human IgE antibody can be used respectively depending which antibody isotype is selected. This detection antibody is labeled, usually by linkage to an enzyme. In the final steps of an ELISA, a substance or agent is added the enzyme-linked detection antibody, the substance or agent is that which the enzyme can convert to some detectable signal. For example, in the case of fluorescence ELISA, when light is shone upon the sample, any antigen/antibody complexes will fluoresce so that the amount of autoantibodies present in the sample can be measured. This is the indirect enzyme-linked immunosorbent assay.

The following is a general standard protocol for setting up and performing an indirect enzyme-linked immunosorbent assay. Using 96-well microtiter plates (Falcon Pro-Bindassay plate 3915; Becton Dickinson, Paramus, N.J.), test wells are coated with one or more desired or target antigen by incubation with 100 μl of a purified CN1A or CN1B or a protein fragment thereof, or an isolated peptide, or a fusion protein described herein (3 g/ml in PBS). The 100 μl aliquots are added to each well and incubated overnight at room temperature, with PBS substituted for the antigen in control wells. After the plates have been washed three times with PBS-Tween, 250 μl of 2% BSA in PBS is added to each well, and the plates are incubated for 1 h at room temperature. The plates are washed three times with PBS-Tween and incubated for 1 h at room temperature with test sera or plasma and control sera or plasma (one high-positive serum or plasma specimen, two negative serum or plasma specimens, and one weak-positive serum or plasma specimen) diluted 1:100 in PBS-Tween-BSA; each serum or plasma specimen is tested in triplicate in antigen-coated wells as well as in antigen control wells. The plate is then assayed (with appropriate controls) for the presence of human auto-antibodies IgG against the desired respective antigen (CN1A or CN1B or a protein fragment or cell/muscle lysate) by incubation for 1 h at room temperature with 100 μl of goat anti-human IgG conjugated with horseradish peroxidase (Bio-Rad, Richmond, Calif.) per well diluted 1:2,000 in PBS-Tween-BSA. After three washes in PBS-Tween, the substrate solution (o-phenylenediamine dihydrochloride; SIGMA-ALDRICH®) is added to each well. The plates are then incubated for 30 min at room temperature in darkness, and the reaction is terminated by the addition of 2N sulfuric acid. The optical density values at 490 nm (OD₄₉₀) are measured in a micro plate ELISA reader. For each serum specimen, mean OD₄₉₀ readings are calculated for the test wells and for the antigen control wells, the latter being subtracted from the former to obtain the net ELISA value.

In one embodiment, an ELISA involving at least one antibody with specificity for the particular desired antigen can also be performed. A known amount of antigen (e.g., 0.5 g of 0.5 g of a CN1A or CN1B or a protein fragment thereof, or an isolated peptide, or a fusion protein described herein) is immobilized on a solid support (usually a polystyrene micro titer plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against CN1A or CN1B or a protein fragment thereof, or an isolated peptide, or a fusion protein described herein and are not derived from the subject are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., blood, serum or plasma) from a patient is mixed with a known amount of desired antigen (e.g., CN1A or CN1B or a protein fragment thereof) together with the horseradish peroxidase labeled antibodies and the mixture is then are added to coated wells to form competitive combination. After incubation, if the autoantibody level is high in the sample, a complex of autoantibodies-antigen-labeled antibody will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the autoantibody level is high in the sample. If there is no autoantibody or little autoantibodies present in the sample, a different complex in formed, the complex of solid support bound antibodies-antigen-labeled antibody. This complex is immobilized on the plate and is not washed away in the wash step. Subsequent incubation with TMB will produce much color change. Such a competitive ELSA test is specific, sensitive, reproducible and easy to operate.

In one embodiment, the reverse-sandwich (RS) ELISA is used (Miyazawa H, et. al, J Allergy Clin Immunol. 1988; 82:407-413), wherein the autoantibody of interest, in the methods and assays described herein, the autoantibodies against CN1A or CN1B or a protein fragment thereof, or an isolated peptide, or a fusion protein described herein, is sandwiched by respective antigen; one antigen is affixed to a surface and the second antigen is soluble and tagged. This method is also known as the double-antigen sandwich method.

The following is a general standard protocol for setting up and performing a RS-ELISA. A 0.1 ml quantity of CN1A or CN1B or a protein fragment thereof, or an isolated peptide, or a fusion protein described herein (0.5 μg/ml) plus bovine serum albumin (BSA; 25 μg/ml) in 0.5 M NaCl-0.1% NaN₃-0.05 M sodium carbonate (pH 9.6) is added to wells of Maxisorp microplates (Nalge Nunc, Copenhagen, Denmark). The plates are incubated overnight at 4° C. for antigen immobilization. After the wells are washed test sera diluted 1:4, 1:40, and 1:400 with FBS-PBST (10% [vol/vol] fetal bovine serum [FBS], 0.1% NaN₃-phosphate-buffered saline [PBS]-0.05% Tween 20 [PBST]) are added, and the plates are incubated for 60 min at room temperature. Seven threefold serial dilutions of a reference serum are used. After another wash, biotinylated respective desired antigen (0.05 μg/ml) in FBS-PBST is then added to the wells, and the reaction is allowed to take place for 60 min at room temperature. The wells are washed again, streptavidin-conjugated β-d-galactosidase (diluted 1:50,000 in PBST containing 1% BSA) is added, and the plates are incubated for 60 min at room temperature. After another wash, 0.2 mM 4-methylumbelliferyl-β-d-galactoside in 0.1M NaCl-1 mM MgCl₂-0.1% BSA-0.1% NaN3-0.01M sodium phosphate (pH 7.0) is added. The wells are sealed with tape, and the plates are immersed in 37° C. water for 60 min. Finally, 0.1 ml of 0.1M glycine-NaOH (pH 10.2) is added to each well to stop the enzyme reaction. The fluorescence units (FU) in each well are measured with a Fluoroskan II apparatus (Flow Laboratories, Rockville, Md.). The antibody isotype concentrations of the test sera or plasma are calculated from the titration curve of the respective reference serum or plasma with known antibody isotype units per milliliter.

In one embodiment, the levels of autoantibody isotypes in a blood sample are detected by a lateral flow immunoassay test (LFIA), also known as the immunochromatographic assay, or strip test. LFIAs are a simple device intended to detect the presence (or absence) of a target autoantibody isotypes in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test it encounters a coloured reagent which mixes with the sample and transits the substrate encountering lines or zones which have been pretreated with an antibody or antigen. Depending upon the autoantibody isotype present in the sample the coloured reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water samples etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be use on site in the field.

LFIA tests can be operated as either competitive or sandwich assays. Sandwich LFIAs are similar to sandwich ELISA.

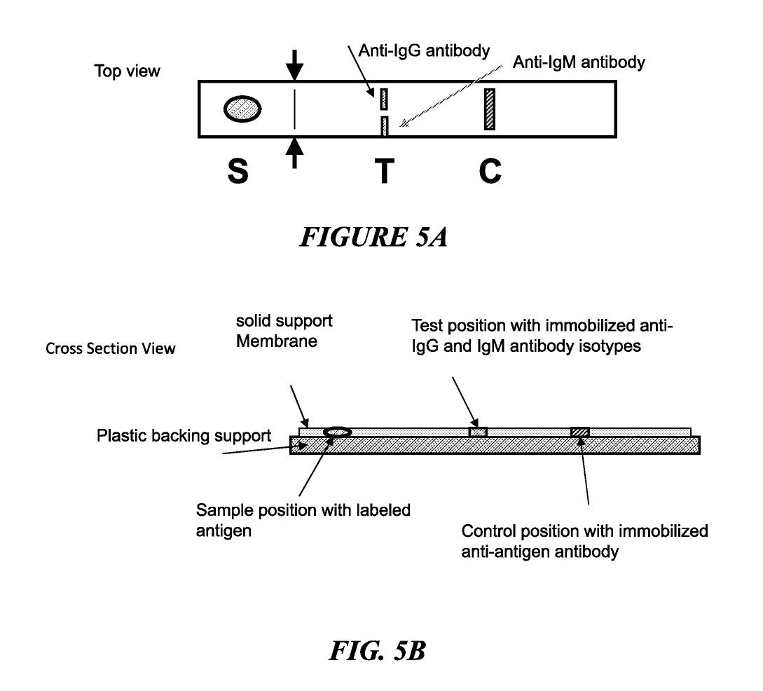

A typical test strip consists of the following components: (1) sample application area comprising an absorbent pad (i. e. the matrix or material) onto which the test sample is applied; (2) conjugate or reagent pad—this contains antibodies or antigen depending on whether the tested entity is an autoantibody isotype or a biomarker (i.e., an antigen), the antibodies or antigen is usually colloidal gold particles, or latex microspheres; test results area comprising a reaction membrane—typically a hydrophobic nitrocellulose or cellulose acetate membrane onto which anti-antigen antibodies are immobilized in a line across the membrane as a capture zone or test line (a control zone may also be present, containing antibodies specific for the conjugate antibodies); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly. FIGS. 5 and 6 show the various components and embodiments of several test strips.

In some embodiments, the lateral flow immunoassay is a double antibody sandwich assay, a competitive assay, a quantitative assay or variations thereof.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teaching of these “dip stick” technology for the detection of auto antibodies.

In one embodiment of any one of assay described herein, the detection antibody used is detectably labeled. As used herein “detectably labeled”, includes antibodies that are labeled by a measurable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, HIS, or biotin.

In one embodiment, the detectable label is a dye. A “dye” refers to a substance, compound or particle that can be detected, particularly by visual, fluorescent or instrumental means. A dye can be, for example, but not limited to, a pigment produced as a coloring agent or ink, such as Brilliant Blue, 3132 Fast Red 2R and 4230 Malachite Blue Lake, all available from Hangzhou Hongyan Pigment Chemical Company, China. The “dye” can also be a particulate label, such as, but not limited to, blue latex beads or gold particles. The particulate labels may or may not be bound to a protein, depending upon if it is desired for the particles to move in the test strip or not. If the particles are to be immobilized in the test strip, the particles may be conjugated to a protein, e. g. the anti-antigen antibody, which in turn is bound to the test strip by either physical or chemical means.

Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif.

In one embodiment of any one of the assays described herein, the detection antibody is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes contemplated for use to detectably label the antibodies include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In other embodiments, the detection antibody is labeled with a fluorescent compound. When the fluorescently labeled antibody is exposed to light of the proper wavelength, its presence can then be detected due to fluorescence. Among the most commonly used fluorescent labeling compounds are CY dyes, fluorescein isothiocyanate, rhodamine, phycoerytherin, phycocyanin, allophycocyanin, o-phthaldehyde and fluorescamine.

A detection antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

A detection antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem. 22:895-904. These references are hereby incorporated by reference in their entirety.

Other techniques can be used to detect the autoantibody isotypes in a sample. One such technique is Western blotting (Towbin et at., Proc. Nat. Acad. Sci. 76:4350 (1979)); another is an adaptation of the Western blot, the dot blots. In the Western blots, a CN1A or CN1B or a protein fragment thereof, or an isolated peptide, or a fusion protein described herein can be dissociated with detergents and heat, and separated on an SDS-PAGE gel before being transferred to a solid support, such as a nitrocellulose membrane. The membrane is incubated with a sample suspected of containing autoantibodies against a CN1A or CN1B or a protein fragment thereof, or an isolated peptide, or a fusion protein respectively. The membrane is then washed to remove unbound proteins and proteins (including antibodies) with non-specific binding. Detectably labeled enzyme-linked secondary or detection antibodies can then be used to detect and assess the amount of autoantibodies in the sample tested. The intensity of the signal from the detectable label corresponds to the amount of enzyme present, and therefore the amount of auto-antibodies against respective antigen. Levels can be quantified, for example by densitometry.

In some embodiments, the immunoassays operate on a purely qualitative basis. However it is possible to measure the intensity of the test line to determine the quantity of autoantibody isotypes in the blood sample when using an immunoassay such a LFIA. Implementing a magnetic immunoassay (MIA) in the lateral flow test form also allows for getting a quantified result.

In one embodiment, the detection of autoantibody isotype(s) against a CN1A or CN1B or a protein fragment thereof, or an isolated peptide, or a fusion protein described herein is considered positive when the immunoassay signal is at least 5% over that of an control immunoassay signal obtained in the absence of any antibody against the respective antigen thereof or in the presence of a non-related antibody or non-related antibody isotype.

By a “non-related antibody” means an antibody that does not bind or is not reactive against a CN1A or CN1B or a protein fragment thereof, or an isolated peptide, or a fusion protein described herein.

In another embodiment, the control immunoassay signal is that obtained with the serum of non-IBM healthy subject, these subjects do not have the clinical features of the disease. In another embodiment, these non-IBM healthy subjects do not have any inflammatory conditions. In another embodiment, the control immunoassay signal is the average value obtained for a population of non-IBM healthy subjects. A population is at least 25 non-IBM healthy subjects, preferably more.

The embodiments described in this disclosure are not intended to be exhaustive or to limit the disclosure to the precise embodiments disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present technology. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

Definitions of common terms used herein can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006 and Current Protocols in Protein Sciences 2009, Wiley Intersciences, Coligan et al., eds.

Unless otherwise stated, the embodiments described herein were performed using standard procedures, as described, for example in The ELISA guidebook, Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

This technology is further illustrated by the following examples which should not be construed as limiting.

### EXAMPLES

### Example 1

### Materials and Methods

Patients and Blood Samples.

All patient samples were collected after informed written consent was obtained and under protocols approved by the Partners Human Research Committee Institutional Review Board (IRB) overseeing Brigham and Women's Hospital human research activities.

Plasma and serum samples from 205 patients were studied (49 with IBM, 156 without IBM: 36 with dermatomyositis (DM), 9 with muscular dystrophy, 13 with myasthenia gravis, 13 with necrotizing myopathy, 28 with polymyositis, 23 with other myopathies, and 34 healthy volunteers). Diagnostic criteria for IBM was European Neuromuscular Centre (ENMC) criteria for probable (N=11) or definite IBM (N=38),⁸ except for 6 patients who did not meet the ENMC requirement of “sporadic” disease because they had an affected relative, as previously noted.⁵ Diagnostic criteria for PM were as previously used.⁹ Diagnostic criteria for necrotizing myopathy included an immunoresponsive myopathy with muscle biopsy demonstrating multifocal myofiber necrosis in the absence of endomysial inflammation and invasion of non-necrotic myofibers. Criteria for DM were the presence of a characteristic skin rash, subacute predominantly proximal weakness, and muscle biopsy showing perifascicular atrophy or perivascular and perimysial inflammation without endomysial inflammation.

Immunoblots and Blocking Experiments.

Immunoblots of whole muscle lysates (25-50 μg, amount constant for all lanes within a given gel), recombinant cN1A (70 ng), and human IgM (cat#009-000-012, Jackson ImmunoResearch, West Grove, Pa.), IgA (cat#009-000-011), and IgG (Cat#009-000-003) were performed with human plasma or sera at 1:1000 dilution. Human plasma and sera studies used secondary antibody goat anti-human IgM-HRP (Pierce Biotechnology, Rockford, Ill., cat#31415), anti-human IgA-HRP (cat#31417), and anti-human IgG-HRP (cat#31410).

Enzyme-Linked Immunoassays (ELISAs)

Enzyme-linked immunoassays (ELISAs) were constructed by coating Nunc 96-well plates with 500 ng per well of recombinant N-terminal His-tagged cN1A/NT5C1A protein (NCBI RefSeq NP_115915.1; GenScript USA Inc. Piscataway, N.J.).

Machine Learning

Machine learning methods were implemented using the software package Orange.¹⁰ Training sets of ⅔ the data were used to build the algorithms and of ⅓ the data to test the algorithms, using random sampling and 10 repetitions of the data.

Results

Presence of serum IgM and IgA anti-cN1A autoantibodies in IBM

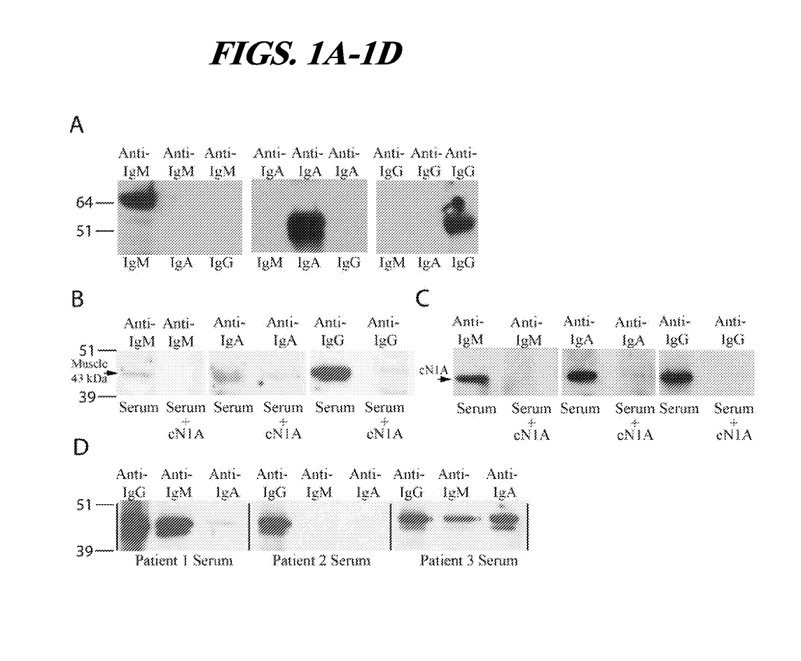

The selectivity of secondary antibodies was confirmed through control experiments with combinatorial probing of purified human IgM, IgA, and IgG with anti-IgM, anti-IgA, and anti-IgG HRP-conjugated antibodies in western blots (FIG. 1A). Skeletal muscle lysates were then probed with human sera and autoantibody isotypes detected through the use of these selective secondary antibodies. These studies showed the presence of IgM and IgA autoantibodies in addition to the previously demonstrated IgG autoantibodies, directed against an approximately 43 kDa muscle protein. Blocking experiments in which patient sera were preabsorbed with recombinant cN1A prior to blotting of skeletal muscle lysates demonstrated the loss of 43 kDa band reactivity to IgM and IgA, confirming that the IgM and IgA autoantibodies against this 43 kDa protein were indeed directed against cN1A (FIG. 1B). Similar blocking experiments blotting preabsorbed sera against denatured recombinant cN1A also showed loss of reactivity, indicating patient serum autoantibodies bound to recombinant cN1A in solution as well as denatured cN1A on membranes (FIG. 1C). Lastly, different patient sera contained various patterns of IgM, IgA, and IgG autoantibody reactivity directed against recombinant cN1A (FIG. 1D).

IgM, IgA, and IgG ELISAs

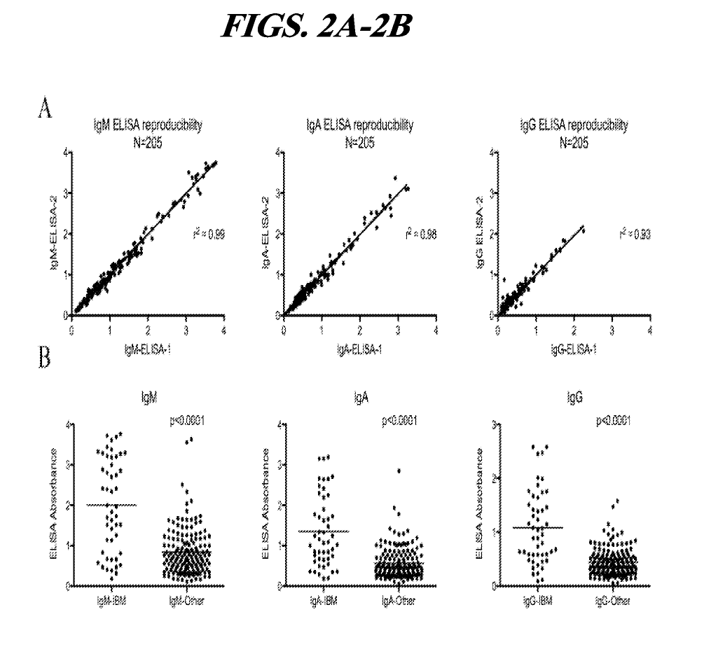

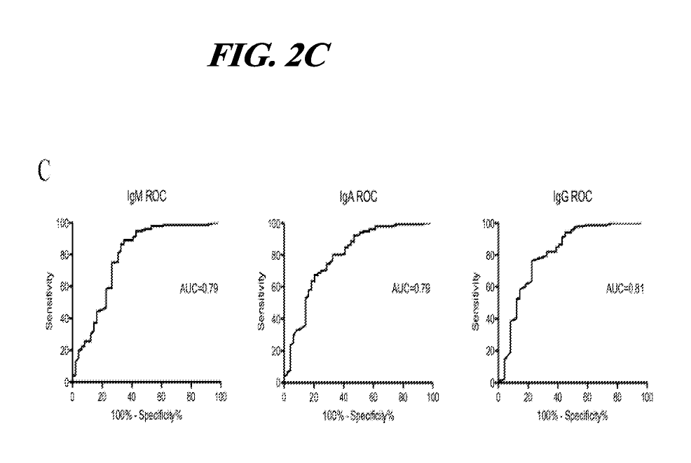

To establish a high throughput method for antibody screening, individual ELISA assays to detect IgM, IgA, and IgG anti-cN1A antibodies were developed. These assays had high reproducibility (FIG. 2A; r2=0.98-0.99 for all). Analysis of 205 patient samples (49 with IBM, 156 non-IBM samples) demonstrated significant group mean differences for all isotypes (p=<0.0001 for each of IgM, IgA, and IgG; FIG. 2B). Receiver operating characteristic analysis showed high area under the curve (0.79 to 0.81), indicating that autoantibody isotype thresholds might serve as robust diagnostic classifiers (FIG. 2C). The comparative diagnostic performances of the ELISAs were similar, with minor superiority of IgM isotype testing (Table 1). For example, threshold tests resulted in sensitivity/specificity/accuracy of 51%/94%/84% with IgG testing and 51%/96%/85% with IgM testing. Depending on choices for cutoffs, these approaches yielded sensitivities ranging from 37-59% and specificities ranging from 90-99%.

Diagnostic Utility of Combination Classifiers

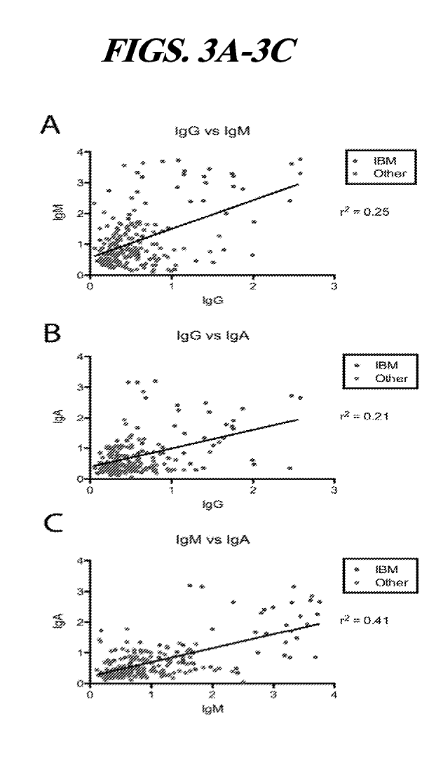

The correlation of IgM and IgA with IgG anti-cN1A autoantibodies was only modest (r²=0.21 to 0.41), indicating that anti-cN1A autoantibody isotypes have separate utility for diagnosis (FIG. 3). IgM, IgA, and IgG autoantibodies were frequently detected independently of each other, so that some patients had high levels of one antibody but low levels of another. For example, 4 IBM patients (8%) had very high IgA levels (>2.5 absorbance units; AU) but low IgG levels (<1 AU), and another 3 (6%) patients had very high IgM but neither high IgA or high IgG levels. These differences indicated a potential role for combination diagnostic classifiers, in which consideration of all 3 autoantibodies are used for diagnostic prediction.

Simple combination thresholding methods improved testing based on the detection of IgG autoantibodies alone (e.g., the sensitivity of 33% reported in⁶). For example, a test based on IgM>1.9 or IgA>1.15 or IgG>1.3 absorbance units improved sensitivity/specificity/accuracy to 73%/91%/87%.

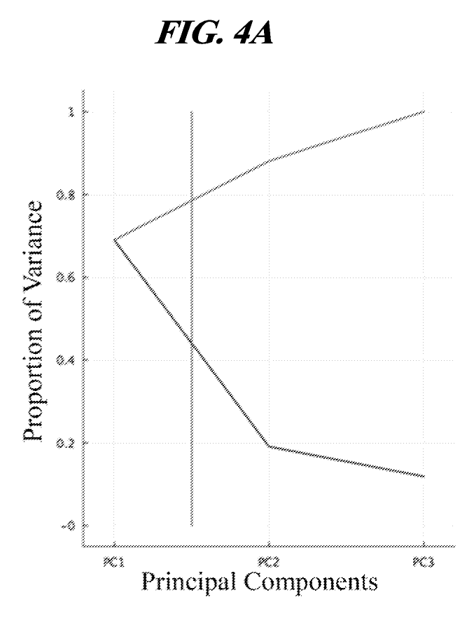

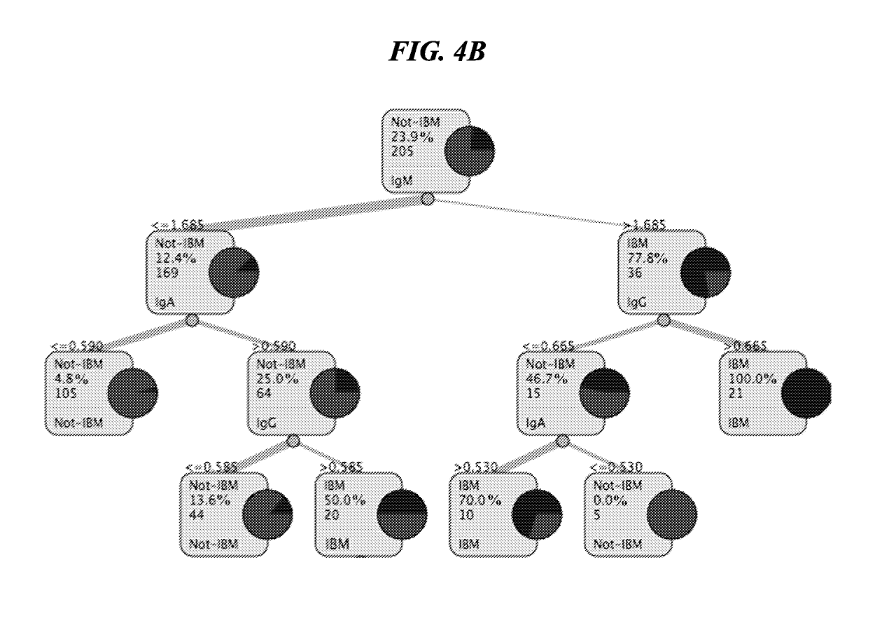

More complex combination approaches involve machine learning techniques, in which diagnostic classifiers are learned from the data. A principal components analysis indicated that almost 80% of variance was due to IgM autoantibody levels (FIG. 4A). An optimized but partially biased approach using classification trees trained on the entire data set of 205 samples demonstrated a sensitivity of 78% and specificity of 92% (FIG. 4B). Unbiased machine learning models including classification trees, naïve Bayes classifiers, and support vector machines, using training/testing datasets demonstrated little improvement upon simple thresholding approaches, with best sensitivity/specificity/accuracy of 55%/96%/87% (Table 1).

### Example 2

### Exemplary Diagnostic Semi-Quantitative Lateral Flow Immunoassay Test Strips

The levels of auto-antibodies described herein can also be determined using test strips as illustrated in FIGS. 5A-6. In the test strip, the membrane is divided into three separate regions: a sample (S) position at one end of the membrane, a test (T) position located at the middle of the membrane, and a control (C) position found at the opposite end the membrane (FIG. 5A). Located at S is an excess quantity of dehydrated desired antigen. The desired antigen can be a CN1A, a CN1B isoform, or peptide thereof, or an isolated peptide or fusion protein described herein. The desired antigen can be conjugated to colloidal gold beads or latex beads for visualization purposes. At T, there is an excess quantity of anti-IgG antibodies and an excess quantity of anti-IgM antibodies are immobilized on the membrane. At C, there is another immobilized anti-IgG antibody and anti-IgM antibodies that are reactive to the desired antigen (FIG. 5A).

The excess quantity of dehydrated desired antigen at S position is such that when a sample (e.g. serum) is applied at S, autoantibody-antigen complexes are formed as well as there is free antigen are still available to bind the immobilized anti-IgG at position C.

The S position is where a sample of serum is applied. The arrowheads delineate the boundary limit that the sample serum should not cross on the membrane when applying the serum to the membrane. The water in the serum rehydrates the desired antigen. An antibody-antigen complex is produced when the autoantibody reactive to forms a complex with the rehydrated desired antigen. A mixture of the autoantibody-antigen complexes and non-complexed antigen move by capillary action away from position S towards position T and C.

Upon arrival at the T position, the autoantibody-antigen complex will bind the immobilized anti-IgG or anti-IgM antibody and be immobilized at the T position. The localized concentration of autoantibody-antigen complex that is colloidal gold or latex bead labeled will become visible as a colored line at the T position (FIG. 6, middle test strip). The greater the amount of autoantibodies in the sample, the broader the visible band at T. When the area remains clear at the T position, this means that there no or below detectable levels of autoantibodies (FIG. 6, far left test strip). At the C position, the free and labeled antigen originating from position S will be bound and captured by the immobilized anti-IgG or anti-IgM that is reactive to the desired antigen. This will in turn result in a concentration of a colloidal gold or latex bead labeled at the C position and will become visible as colored line at the C position. The C position result serves as a test control that there is functional in the test strip and should always be present (FIG. 6).

The test strip can be designed in a form of a dipstick test strip (FIG. 5B). As a dipstick test strip, the strip is dipped into a sample of serum at the S position end with sample level not to exceed the boundary limit. The strip is then laid horizontally with the membrane surface facing up on a flat surface. A fixed amount of time is given for the rehydration of the desired antigen, capillary action, and antibody binding reaction to take place. At the end of the fixed time, there should be visible bands at the C position and depending on the level of auto-anti-antibody, there may or may not be a visible band at the T position (FIG. 6).

### REFERENCES

1. Greenberg S A, Sanoudou D, Haslett J N et al. Molecular profiles of inflammatory myopathies. Neurology. 2002; 59:1170-1182
2. Greenberg S A, Bradshaw E M, Pinkus J L et al. Plasma cells in muscle in inclusion body myositis and polymyositis. Neurology. 2005; 65:1782-1787
3. Bradshaw E M, Orihuela A, McArdel S L et al. A local antigen-driven humoral response is present in the inflammatory myopathies. J Immunol. 2007; 178:547-556
4. Salajegheh M, Lam T, Greenberg S A. Autoantibodies against a 43 KDa muscle protein in inclusion body myositis. PLoS One. 2011; 6:e20266
5. Larman H B, Salajegheh M, Nazareno R et al. Cytosolic 5′-Nucleotidase 1A Autoimmunity in Sporadic Inclusion Body Myositis. Ann Neurol. 2013; 73:408-418
6. Pluk H, van Hoeve B J, van Dooren S H et al. Autoantibodies to cytosolic 5′-nucleotidase IA in inclusion body myositis. Ann Neurol. 2013; 73:397-407
7. Needham M, James I, Corbett A et al. Sporadic inclusion body myositis: phenotypic variability and influence of HLA-DR3 in a cohort of 57 Australian cases. Journal of neurology, neurosurgery, and psychiatry. 2008; 79:1056-1060
8. Badrising U A, Maat-Schieman M, van Duinen S G et al. Epidemiology of inclusion body myositis in the Netherlands: a nationwide study. Neurology. 2000; 55:1385-1387
9. Chahin N, Engel A G. Correlation of muscle biopsy, clinical course, and outcome in PM and sporadic IBM. Neurology. 2008; 70:418-424
10. Curk T, Demsar J, Xu Q et al. Microarray data mining with visual programming. Bioinformatics. 2005; 21:396-398

TABLE 1
Diagnostic performance of single immunoglobulin (Ig) threshold,
combination Ig threshold, and machine learning assays. A combination
assay of thresholds for all 3 imunoglobulin subtypes has improved
sensitivity at little cost in specificity. Machine learning
methods applied to training/test (66%/33%) data.

| Approach | Sensitivity | Specificity | Accuracy | AUC |
|---|---|---|---|---|
| Single Ig Subytpe | | | | |
| IgM > 1.9 | 51% | 96% | 85% | 0.79 |
| IgA > 1.2 | 49% | 94% | 83% | 0.79 |
| IgG > 0.9 | 51% | 94% | 84% | 0.81 |
| Combination Ig Subytpes | | | | |
| IgM > 1.9, IgA > 1.1, | 73% | 92% | 87% | 0.82 |
| IgG > 1.3 | | | | |
| Machine Learning | | | | |
| Classification Tree | 49% | 90% | 80% | 0.67 |
| Naïve Bayes | 55% | 96% | 86% | 0.83 |
| Support Vector Machine | 53% | 97% | 87% | 0.84 |

AUC = area under curve.

TABLE 2
Diagnostic performance of combination immunoglobulin (Ig)
isotypes with different thresholds for each isotypes.

| Combination Ig Isotypes | Sensitivity | Specificity | Accuracy |
|---|---|---|---|
| IgM > 1.90, IgA > 1.15, | 76% | 91% | 87% |
| IgG > 1.30 | | | |
| (IgM > 1.4, IgA > 1.20); or | 71% | 87% | 83% |
| (IgM > 1.4, IgG > 1.30) | | | |
| (IgG > 0.9, IgA > 1.00); or | 71% | 90% | 85% |
| (IgG > 0.9, IgM > 1.15) | | | |
| IgM > 1.90, IgA > 1.40 | 69% | 87% | 82% |

Various thresholds selected for IgM for this study are >1.90; >1.4; and >1.15.
Various thresholds selected for IgG for this study are >0.9; >1.3.
Various thresholds selected for IgA for this study are >1.00 >1.20; >1.15; and >1.40.
Sensitivity = number of positive IBM/number of total IBM samples
Specificity = number of negative non-IBM/number of total non-IBM samples
Accuracy = [number of positive IBM + number of negative non-IBM]/[number of total IBM + number of total non-IBM]

1. An assay comprising
a. providing a biological sample from a subject in need of diagnosing, and
b. measuring for a detectable presence or an absence or a level of at least two antibody isotypes that are reactive against a cytosolic 5′-nucleotidase 1A protein (cN1A) or a protein fragment thereof, or a cytosolic 5′-nucleotidase 1B (cN1B) protein or isoforms thereof or a protein fragment thereof for diagnosing or detecting inclusion body myositis (IBM) in a subject wherein the antibodies isotypes are selected from the group selected from IgG, IgM, IgA, IgD, and IgE.

2. The assay of claim 1, further comprising diagnosing the likelihood of IBM based on the detectable presence or the level of the at least two antibody isotypes selected in step b.

3.-5. (canceled)

6. The assay of claim 1, wherein the subject in need of diagnosing has trouble with gripping, or has frequent stumbles, or has trouble swallowing due to weakness of the swallowing muscles.

7. The assay of claim 1, wherein the subject has not had to an electromyogram and/or a muscle biopsy.

8.-12. (canceled)

13. The assay of claim 2, wherein the at least two isotypes are IgG and IgM; IgG and IgA; IgG, IgA and IgM; IgA and IgM; IgG and IgD; IgG and IgE; IgD and IgM; IgE and IgM; IgE and IgA; IgD and IgA; IgE, IgM and IgA; IgD, IgM and IgA; IgD, IgE and IgM; IgD, IgE and IgA; and IgD, IgE, IgA and IgM.

14.-17. (canceled)

18. The assay of claim 2, wherein IgM is one of the at least two isotypes selected.

19. The assay of claim 2, wherein IgM is one of the at least two isotypes selected and IgG is not selected.

20. The assay of claim 2, wherein the detectable presences of the at least two antibody isotypes selected indicate the likelihood of IBM in the subject.

21. The assay of claim 2, further comprising comparing the level of the at least two antibody isotypes selected to respective antibody isotype reference levels to determine the likelihood of IBM in the subject.

22. The assay of claim 2, wherein the level of the at least two isotypes that are at least 5% over that of respective antibody isotype reference levels indicate the likelihood of IBM.

23. The assay of claim 22, wherein the respective antibody isotype reference levels are selected from (a) the levels of the selected antibody isotypes that are reactive against the same cN1A, cN1B or protein fragment thereof in a biological sample of a healthy subject not having IBM, wherein the biological samples are the same for both the healthy subject and subject in need of diagnosis; (b) the average levels of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis; (c) the average levels of the selected antibody isotypes that are reactive against the same cN1A, cN1B or fragment thereof in a plurality of biological samples from a population of healthy subjects not having IBM, wherein the biological samples are the same for both the healthy subject and subject in need of diagnosis; and (d) the normalized levels of the selected antibody isotypes that are reactive against the same cN1A, cN1B or fragment thereof in a biological sample of a healthy subject not having IBM, wherein the normalization is performed against a level of albumin in the biological sample of a healthy subject not having IBM, wherein the biological samples are the same for both the healthy subject and subject in need of diagnosis.

24.-27. (canceled)

28. The assay of claim 1, wherein the measuring method comprises the steps of
i) contacting the blood sample from the subject with a cN1A, cN1B or protein fragment thereof;
ii) forming an antibody-protein complex between the antibody isotype present in the blood sample with the cN1A, cN1B or protein fragment thereof;
iii) washing to remove any unbound antibody;
iv) adding at least two detection antibodies that are labeled and are respectively reactive to the at least two antibody isotypes selected for the assay from the
v) blood sample of the subject in order to detect the antibody-protein complex formed in step ii);
vi) washing to remove any unbound labeled detection antibodies; and
vii) converting the label of the at least two detection antibodies to detectable signals, wherein the a detectable signal for each of the at least two detection antibodies indicates the presence or level of respective anti-cN1A or anti-cN1B autoantibody isotype in the biological sample of the subject.

29. The assay of claim 1, wherein the measurement method is an immunoassay.

30.-36. (canceled)

37. The assay of claim 1 further comprising selecting the subject for treatment of IBM without having to perform an electromyogram and/or a muscle biopsy on the subject when there is detectable presence or the level of the at least two antibody isotypes selected in step b.

38. The assay of claim 1, further comprising selecting the subject for treatment of IBM without having to perform an electromyogram and/or a muscle biopsy on the subject when the level of the at least two isotypes selected in step b are at least 5% over that of respective antibody isotype reference levels.

39. An assay comprising:
a. measuring for an absence or a presence or a level of at least two antibody isotypes that are reactive against a cytosolic 5′-nucleotidase 1A protein (cN1A), or a cytosolic 5′-nucleotidase 1B (cN1B) protein or isoforms thereof in at least two biological samples obtained from a subject at different times for a prognosis evaluation of the subject with IBM or for evaluating the efficacy of a treatment in the subject with IBM, wherein the different times are a first time point and at least second time point, wherein the second time point is after the first time point, wherein the patient has inclusion body myositis (IBM) and is being treated for IBM, and wherein the antibodies isotypes are selected from the group selected from IgG, IgM, IgA, IgD, and IgE; and
b. evaluating the prognosis of the subject or the treatment efficacy based on the detectable presence or the level of the at least two antibody isotypes selected in measuring step a.

40.-43. (canceled)

44. A kit for performing the assay of claim 1 comprising:
a. a CN1A or CN1B or a protein fragment thereof, or an isolated peptide of CN1A or CN1B, or a fusion protein of CN1A or CN1B;
b. at least two different detection antibodies, wherein the detection antibody is specific for the antibodies of a patient and specific for at least two of antibody isotypes selected from the group selected from IgG, IgM, IgA, IgD, and IgE.

45.-46. (canceled)

47. A system comprising:
a. a measuring module measuring autoantibody isotype information comprising at least two detectable signal from an immunoassay indicating the presence or level of autoantibody isotype that are reactive to at least a CN1A or CN1B or isoform thereof, peptide fragment thereof, the autoantibody isotypes are from a biological sample obtained from a patient, wherein the antibody isotypes are selected from the group selected from IgG, IgM, IgA, IgD, and IgE, and wherein each signal level correspond to an antibody isotype;
b. a storage module configured to store data output from the measuring module;
c. a comparison module adapted to compare the data stored on the storage module with at least two reference levels, and to provide a retrieved content, wherein reference levels are the reference levels for the respective antibodies isotypes measured and are selected from the group selected from IgG, IgM, IgA, IgD, and IgE; and
d. an output module for displaying the retrieved content for the user, wherein the retrieved content, the presence of detectable presence or amount of at least autoantibody isotype reactive against the protein or peptide or fusion protein used of step a, indicates that the patient has IBM or has a relapse of IBM.

48.-50. (canceled)

51. A method of treatment of inclusion body myositis (IBM) in a subject in need thereof comprising:
a. Performing an assay of claim 1;
b. Selecting the subject for treatment if there is a detectable signal for each of the at least two detection antibody isotype selected for the assay; and
c. Administering an immunosuppressive therapy to the subject.

51.-53. (canceled)

54. The method of claim 51, further comprising comparing the levels of the at least two antibody isotypes at a second time point, wherein a decrease in the level of the antibody isotypes taken at the second time point indicates that the therapy is effective, wherein an increase in the level of the antibodies taken at the second time point are approximately the same indicates that the treatment is not effective, and wherein when the level of antibodies in the second time point decreases to below a detection limit indicates that the subject is in remission.
